SEQUENCE LISTING (27 sequences)

SEQ ID NO: 1 - protein, 9 aa, Artificial sequence
  Synthetic peptide
XIRYSGHSL

SEQ ID NO: 2 - DNA, 2480 bp, Homo sapiens
gacgctctgtgccttcggaggtctttctgcctgcctgtcctcatgcctctcctcctcttgctgctcctgctgccaagccccttacacccccaccccatctgtgaggtctccaaagtggccagccacctagaagtgaactgtgacaagaggaatctgacagcgctgcctccagacctgccgaaagacacaaccatcctccacctgagtgagaacctcctgtacaccttctccctggcaaccctgatgccttacactcgcctcactcagctgaacctagataggtgcgagctcaccaagctccaggtcgatgggacgctgccagtgctggggaccctggatctatcccacaatcagctgcaaagcctgcccttgctagggcagacactgcctgctctcaccgtcctggacgtctccttcaaccggctgacctcgctgcctcttggtgccctgcgtggtcttggcgaactccaagagctctacctgaaaggcaatgagctgaagaccctgcccccagggctcctgacgcccacacccaagctggagaagctcagtctggctaacaacaacttgactgagctccccgctgggctcctgaatgggctggagaatctcgacacccttctcctccaagagaactcgctgtatacaataccaaagggcttttttgggtcccacctcctgccttttgcttttctccacgggaacccctggttatgcaactgtgagatcctctattttcgtcgctggctgcaggacaatgctgaaaatgtctacgtatggaagcaaggtgtggacgtcaaggccatgacctctaacgtggccagtgtgcagtgtgacaattcagacaagtttcccgtctacaaatacccaggaaaggggtgccccacccttggtgatgaaggtgacacagacctatatgattactacccagaagaggacactgagggcgataaggtgcgtgccacaaggactgtggtcaagttccccaccaaagcccatacaaccccctggggtctattctactcatggtccactgcttctctagacagccaaatgccctcctccttgcatccaacacaagaatccactaaggagcagaccacattcccacctagatggaccccaaatttcacacttcacatggaatccatcacattctccaaaactccaaaatccactactgaaccaaccccaagcccgaccacctcagagcccgtcccggagcccgccccaaacatgaccaccctggagcccactccaagcccgaccaccccagagcccacctcagagcccgcccccagcccgaccaccccggagcccaccccaatcccgaccatcgccacaagcccgaccatcctggtgtctgccacaagcctgatcactccaaaaagcacatttttaactaccacaaaacccgtatcactcttagaatccaccaaaaaaaccatccctgaacttgatcagccaccaaagctccgtggggtgctccaagggcatttggagagctccagaaatgacccttttctccaccccgacttttgctgcctcctccccctgggcttctatgtcttgggtctcttctggctgctctttgcctctgtggtcctcatcctgctgctgagctgggttgggcatgtgaaaccacaggccctggactctggccaaggtgctgctctgaccacagccacacaaaccacacacctggagctgcagaggggacggcaagtgacagtgccccgggcctggctgctcttccttcgaggttcgcttcccactttccgctccagcctcttcctgtgggtacggcctaatggccgtgtggggcctctagtggcaggaaggaggccctcagctctgagtcagggtcgtggtcaggacctgctgagcacagtgagcattaggtactctggccacagcctctgagggtgggaggtttggggaccttgagagaagagcctgtgggctctcctattggaatctagttgggggttggaggggtaaggaacacagggtgataggggaggggtcttagttcctttttctgtatcagaagccctgtcttcacaacacaggcacacaatttcagtcccagccaaagcagaaggggtaatgacatggacttggcggggggacaagacaaagctcccgatgctgcatggggcgctgccagatctcacggtgaaccattttggcagaatacagcatggttcccacatgcatttatgcacagaagaaaatctggaaagtgatttatcaggatgtgagcactcgttgtgtctggatgttacaaatatgggtggttttattttctttttccctgtttagcattttctagttttcttatcaggatgtgagcactcgttgtgtctggatgttacaaatatgggtggttttattttctttttccctgtttagcattttctagttttccactattattgtatattatctgtataataaaaaataattttagggttggg

SEQ ID NO: 3 - protein, 626 aa, Homo sapiens
MPLLLLLLLLPSPLHPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVRATRTVVKFPTKAHTTPWGLFYSWSTASLDSQMPSSLHPTQESTKEQTTFPPRWTPNFTLHMESITFSKTPKSTTEPTPSPTTSEPVPEPAPNMTTLEPTPSPTTPEPTSEPAPSPTTPEPTPIPTIATSPTILVSATSLITPKSTFLTTTKPVSLLESTKKTIPELDQPPKLRGVLQGHLESSRNDPFLHPDFCCLLPLGFYVLGLFWLLFASVVLILLLSWVGHVKPQALDSGQGAALTTATQTTHLELQRGRQVTVPRAWLLFLRGSLPTFRSSLFLWVRPNGRVGPLVAGRRPSALSQGRGQDLLSTVSIRYSGHSL

SEQ ID NO: 4 - protein, 610 aa, Homo sapiens
HPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVRATRTVVKFPTKAHTTPWGLFYSWSTASLDSQMPSSLHPTQESTKEQTTFPPRWTPNFTLHMESITFSKTPKSTTEPTPSPTTSEPVPEPAPNMTTLEPTPSPTTPEPTSEPAPSPTTPEPTPIPTIATSPTILVSATSLITPKSTFLTTTKPVSLLESTKKTIPELDQPPKLRGVLQGHLESSRNDPFLHPDFCCLLPLGFYVLGLFWLLFASVVLILLLSWVGHVKPQALDSGQGAALTTATQTTHLELQRGRQVTVPRAWLLFLRGSLPTFRSSLFLWVRPNGRVGPLVAGRRPSALSQGRGQDLLSTVSIRYSGHSL

SEQ ID NO: 5 - DNA, 25 bp, Artificial sequence
  Synthetic primer
agaagaattcgctgctctgaccaca

SEQ ID NO: 6 - DNA, 30 bp, Artificial sequence
  Synthetic primer
taagtctagatcagagggcgtggccagagt

SEQ ID NO: 7 - protein, 4 aa, Artificial sequence
  Synthetic peptide
RGDS

SEQ ID NO: 8 - protein, 9 aa, Artificial sequence
  Synthetic peptide
XIRYSGHDL

SEQ ID NO: 9 - protein, 9 aa, Artificial sequence
  Synthetic peptide
SIRYSGHEL

SEQ ID NO: 10 - protein, 7 aa, Artificial sequence
  Synthetic peptide
RYSGHSL

SEQ ID NO: 11 - protein, 100 aa, Artificial sequence
  Synthetic peptide
SWVGHVKPQALDSGQGAALTTATQTTHLELQRGRQVTVPRAWLLFLRGSLPTFRSSLFLWVRPNGRVGPLVAGRRPSALSQGRGQDLLSTVSIRYSGHSL

SEQ ID NO: 12 - protein, 10 aa, Artificial sequence
  Synthetic peptide
VSIRYSGHSL

SEQ ID NO: 13 - protein, 11 aa, Artificial sequence
  Synthetic peptide
TVSIRYSGHSL

SEQ ID NO: 14 - protein, 12 aa, Artificial sequence
  Synthetic peptide
STVSIRYSGHSL

SEQ ID NO: 15 - protein, 13 aa, Artificial sequence
  Synthetic peptide
LSTVSIRYSGHSL

SEQ ID NO: 16 - protein, 14 aa, Artificial sequence
  Synthetic peptide
LLSTVSIRYSGHSL

SEQ ID NO: 17 - protein, 15 aa, Artificial sequence
  Synthetic peptide
DLLSTVSIRYSGHSL

SEQ ID NO: 18 - protein, 16 aa, Artificial sequence
  Synthetic peptide
QDLLSTVSIRYSGHSL

SEQ ID NO: 19 - protein, 17 aa, Artificial sequence
  Synthetic peptide
GQDLLSTVSIRYSGHSL

SEQ ID NO: 20 - protein, 18 aa, Artificial sequence
  Synthetic peptide
RGQDLLSTVSIRYSGHSL

SEQ ID NO: 21 - protein, 19 aa, Artificial sequence
  Synthetic peptide
GRGQDLLSTVSIRYSGHSL

SEQ ID NO: 22 - protein, 20 aa, Artificial sequence
  Synthetic peptide
QGRGQDLLSTVSIRYSGHSL

SEQ ID NO: 23 - protein, 21 aa, Artificial sequence
  Synthetic peptide
SQGRGQDLLSTVSIRYSGHSL

SEQ ID NO: 24 - protein, 22 aa, Artificial sequence
  Synthetic peptide
LSQGRGQDLLSTVSIRYSGHSL

SEQ ID NO: 25 - protein, 23 aa, Artificial sequence
  Synthetic peptide
ALSQGRGQDLLSTVSIRYSGHSL

SEQ ID NO: 26 - protein, 24 aa, Artificial sequence
  Synthetic peptide
SALSQGRGQDLLSTVSIRYSGHSL

SEQ ID NO: 27 - protein, 25 aa, Artificial sequence
  Synthetic peptide
PSALSQGRGQDLLSTVSIRYSGHSL

(19) United States
(12) Patent Application Publication
(10) Pub. No.: US 2009/0181057 A1
(43) Pub. Date: Jul. 16, 2009

(54) Methods and Compositions for the Inhibition of Thrombus Formation

(75) Inventor: Xiaoping Du, Willowbrook, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: THE BOARD OF TRUSTEES OF THE UNIVERSITY OF ILLINOIS, Urbana, IL (US)

(21) Appl. No.: 12/354,525

(22) Filed: Jan. 15, 2009

Related U.S. Application Data
(63) Continuation-in-part of application No. 11/579,291, filed on Jan. 2, 2008.
(60) Provisional application No. 60/568,042, filed on May 4, 2004.

(51) Int. Cl. A61K 9/00; A61K 38/08; A61P 7/02

(52) U.S. Cl. 424/400; 514/15

(57) ABSTRACT

The present invention is directed to compositions comprising inhibitors of GPIb-IX-VWF binding and methods of treating or preventing thrombosis associated with sepsis or endotoxemia in a mammal.

### CROSS REFERENCE TO RELATED APPLICATIONS

The present application is a continuation-in-part of U.S. application Ser. No. 11/579,291, which was filed on Jan. 2, 2008 under 35 U.S.C. §371 as a U.S. national phase application of PCT application no. PCT/US05/14528, which was filed Apr. 27, 2005. The aforementioned PCT application claimed benefit of priority of U.S. Provisional Application No. 60/568,042, which was filed May 4, 2004. The entire text of each of the aforementioned applications is incorporated herein by reference.

### STATEMENT OF GOVERNMENT INTEREST

This invention was made with government support under Grant Number HL62350 awarded by the NIH/NHLBI. The government has certain rights in the invention.

### BACKGROUND

1. Field of the Invention

The present invention is directed to methods and compositions to treat or prevent thrombus formation and treat disorders associated with thrombus formation, such as endotoxemia (i.e., the presence of endotoxins in the blood) and sepsis. More particularly, the present invention provides inhibitors comprising peptides that inhibit the binding of a key factor in the coagulation cascade, von Willebrand factor (VWF), to platelets and inhibit platelet adhesion and thrombus formation. As such, the present invention is generally directed to compositions comprising inhibitors of GPIb-IX-VWF binding and methods of treating or preventing thrombosis associated with sepsis or endotoxemia in a mammal. Compositions of the present invention also inhibit the function of a family of intracellular proteins, named 14-3-3.

2. Background of the Related Art

Blood vessels operate under significant shear stresses that are a function of blood flow shear rate. Frequently, there is damage to small blood vessels and capillaries. When these vessels are damaged, hemostasis is triggered to stop the bleeding. Under typical circumstances, such an injury is dealt with through a sequence of events commonly referred to as the “thrombus formation”. Thrombus formation is dependent upon platelet adhesion, activation and aggregation and the coagulation cascade that culminates in the conversion of soluble fibrinogen to insoluble fibrin clot. Thrombus formation at site of wound prevents extravasation of blood components. Subsequently, wound healing and clot dissolution occurs and blood vessel integrity and flow is restored. Abnormal thrombus formation that causes obstruction of blood vessels is referred to as “thrombosis”.

A key step in the thrombus formation is platelet adhesion. In arteries and capillaries where blood flow shear rate is high, initial platelet adhesion is dependent on the binding of von Willebrand factor (VWF) to its platelet receptor, the glycoprotein (GP) Ib-IX-V complex (GPIb-IX) (Ruggeri, Prog Hemost Thromb 10, 35-68, 1991; Ware, Thromb Haemost 79, 466-478, 1998). VWF binding to GPIb-IX mediates initial platelet adhesion to blood vessel wall, induces platelet activation and firm adhesion, leading platelet aggregation and formation of thrombus. Thus, inhibition of VWF binding to GPIb-IX leads to inhibition of thrombus formation.

GPIb-IX consists of four subunits, GPIbα, GPIbβ, GPIX and GPV. Extracellular domain of GPIba contains binding sites for VWF and thrombin. Binding of VWF to GPIb is regulated by cytoplasmic domain of GPIb-IX. A phosphoserine-dependent intracellular signaling molecule, ζ-form of 14-3-3 protein (Fu et al., Annu Rev Pharmacol Toxicol 40, 617-647, 2000), interacts with the cytoplasmic domain of GPIbα (Du et al., J. Biol Chem 269, 18287-18290, 1994; Du et al., J. Biol Chem 271, 7362-7367, 1996) and this interaction is dependent upon phosphorylation at Serine 609 of GPIbα (Bodnar et al., J. Biol. Chem. 274, 33474-33479, 1999). 14-3-3 is a family of intracellular signaling proteins that specifically recognize intracellular proteins that contains specific serine-phosphorylated 14-3-3 binding motifs. Different 14-3-3 binding proteins may have different sequences. However, these proteins are believed to bind to the same ligand binding pocket in 14-3-3. Thus, binding of one ligand may inhibit binding of a different ligand.

While thrombus formation is an essential mechanism by which unnecessary blood loss is avoided, this system often is dysregulated and leads to the formation of aberrant clots in the vasculature of a mammal. Thrombosis, is the physical condition that manifests when a thrombus is present in the vasculature of an animal. A thrombus (also called clot) is—gel-like or solidified blood formed by polymerized fibrin, platelets, and blood elements trapped by the fibrin-platelet net. While certain authorities imply a difference in the meaning of the terms “blood clot” and “thrombus,” these terms are typically employed interchangeably in the art to mean an aggregation as described above and the terms are used interchangeably herein.

The presence of thrombi in blood vessels can result in and/or from pathologies or treatments such as myocardial infarction, unstable angina, atrial fibrillation, stroke, renal damage, percutaneous translumenal coronary angioplasty, athreosclerosis, disseminated intravascular coagulation, sepsis, pulmonary embolism and deep vein thrombosis. Blood clots also are seen on the surfaces of artificial organs, shunts and prostheses such as artificial heart valves that are implanted into an animal. In addition, certain pathological conditions (such as genetic lack of VWF cleaving protease, ADAMT13) causes spontaneous binding of VWF to platelets resulting in formation of microthrombi in blood vessels leading to thrombotic thrombocytopenic purpura and other microangiopathy. Microangiopathy is a disease of blood vessels in which the walls of very small blood vessels (capillaries) become so thick and weak that they bleed, leak protein, and slow the flow of blood.

In order to combat the deleterious effects of thrombosis, anticoagulants, such as heparin are routinely administered. However, the problem with many existing anti-coagulants is that they fail to block platelet adhesion and aggregation, and can lead to uncontrolled bleeding or other complications. Therefore, there is a constant need to identify new and improved anti-thrombotic drugs.

The present invention is directed to new compositions that may be used as antithrombotics and/or anti-platelet agents.

### SUMMARY OF THE INVENTION

The present invention is directed to peptide-based compositions derived from platelet GPIba C-terminal residues 602-610 and the like to inhibit VWF binding function of GPIb-IX, VWF-mediated platelet adhesion, platelet activation and aggregation, and in vivo thrombus formation. Thus, such compositions may be developed as a new type of anti-thrombotic agents. In particular, the findings of the present invention are based in part on the discovery that 14-3-3 interaction with GPIb-IX is required for the function of GPIb-IX, and thus any inhibitors of this interaction may be used as inhibitors of GPIb-IX and thus used as anti-thrombotic agents. The compositions described herein can be used as inhibitors of intracellular functions of 14-3-3 in cells and in vivo.

Thus, in specific embodiments, the present invention is directed to a composition comprising a myristoylated peptide having an amino acid sequence of, C₁₃H₂₇CONH-SIRYSGHpSL (SEQ ID NO:1 in which the lower case p before serine represents phosphorylation); a fragment of SEQ ID NO:1 that retains a 14-3-3 binding activity, or a conservative variant SEQ ID NO:1 that retains a 14-3-3 binding activity, wherein the myristoyl group is at the C-terminus, or at the N-terminus of the protein. As indicated in preferred embodiments, the peptide is phosphorylated. Preferably, the phosphorylation is on one or more serine/threonine residues of GPIbα. In preferred embodiments, the phosphorylation is at Serine 609. In still other aspects of the invention, the phosphorylated serine residues indicated above are substituted with an aspartic acid or glutamic acid or other composition to simulate the effect of phosphorylation. An exemplary such derivative is a composition comprising the sequence of C₁₃H₂₇CONH-SIRYSGHDL (SEQ ID NO:8). In specific embodiments, the peptide is between about 7 amino acids and about 50 amino acids in length. In other embodiments, the peptide is between about 10 amino acids and about 40 amino acids in length. In preferred embodiments, the contacting a peptide of the invention with platelets, or other type of cells inhibits intracellular function of 14-3-3 to interact with other proteins. In particular aspects, the peptides are used for the inhibition of intracellular function of 14-3-3. Such inhibition may be carried out in vivo or in vitro.

The composition of the present invention is a peptide that inhibits the binding of von Willebrands factor (VWF) to blood platelets, or other cells that express GPIb-IX. In specific embodiments, the peptide inhibits VWF binding to GPIb-IX molecules. In other preferred embodiments, the peptide inhibits VWF binding to platelets. In other embodiments, the peptide inhibits VWF binding to cells expressing GPIb-IX. In particular embodiments, the peptide inhibits GPIb-IX-dependent platelet aggregation.

The peptide compositions of the present invention preferably further comprise a pharmaceutically acceptable carrier, diluent or excipient. In additional embodiments, the compositions of the invention further may comprise an additional agent selected from the group consisting of a fibrinolytic molecule, an anticoagulant and an anti-platelet agent. In particular embodiments, the anticoagulant is selected from the group consisting of a heparin, hirudin or activated protein C. Exemplary fibrinolytic molecules include but are not limited to plasmin or a plasminogen activator. Preferred plasminogen activators include but are not limited to staphylokinase, streptokinase, prourokinase, urokinase, tissue-type plasminogen activator and vampire bat plasminogen activator.

The compositions of the invention may further include a heparin composition. More particularly, the heparin composition is a low molecular weight heparin composition. Low molecular weight heparin compositions are well known to those of skill in the art and include but are not limited to tinzaparin, certoparin, pamaparin, nadroparin, ardeparin, enoxaparin, reviparin, reviparin, dalteparin, and fraxiparin.

The compositions comprising an anti-platelet agent may include an anti-platelet agent selected from the group consisting of ticlopidinem aspirin, clopidrigel or an inhibitor of glycoprotein IIb/IIa function. Other exemplary anti-platelet agents may be selected from the group consisting of Aggrastat™, Aggrenox™, Agrylin™, Flolan™, Integrilin™, Presantine™, Plavix™, Pletal™ and ReoPrO™.

The compositions described herein may be formulated for aerosol, intravenous, oral or topical delivery. The compositions described herein may also be formulated to increase solubility and enhance the entry of the agent into cells. In specific embodiments, the described peptides are formulated as micelles. For example, in some embodiments, the formulation is comprised of PEG₂₀₀₀-DSPE, L-α-phosphatidylcholine (egg PC, Type XI-E) and the peptide mixed at the molar ratio of 45:5:1.

Another aspect of the present invention contemplated method of inhibiting platelet adhesion comprising contacting a sample containing platelets with a composition comprising a peptide derived from GPIbα that binds 14-3-3. In other specific aspects of the invention there is a method of decreasing binding of von Willebrand's factor to platelet cells comprising contacting a biological sample containing said platelet cells with a composition comprising a peptide derived from GPIbα that binds 14-3-3. In preferred embodiments, the contacting is carried out in vivo. In other embodiments, the contacting is carried out in vitro.

The methods described herein may be used as methods of treating or preventing thrombosis in a mammal comprising administering to said mammal a composition of comprising a peptide derived from GPIbα that binds 14-3-3. In exemplary embodiments, the bleeding is in a patient during surgery.

Also encompassed by the present invention is a method of inhibiting thrombosis in a mammal comprising administering to said mammal a composition of comprising a peptide derived from GPIbα that binds 14-3-3, and treating bleeding disorders associated with consumption of platelets and VWF caused by VWF binding to platelets and microthrombus formation. In certain such embodiments, it is contemplated that the mammal is undergoing a procedure during which the mammal's blood is subject to extracorporeal circulation and said administering comprises admixing said composition with the extracorporeal circulating blood in an amount effective to inhibit platelet aggregation in said circulating blood. More particularly, the mammal is subjected to extracorporeal circulation during transplant surgery, abdominal surgery, vascular surgery, or cardiopulmonary bypass surgery. In other embodiments, it is contemplated that the thrombosis is associated with atherosclerosis, myocardial infarction, unstable angina, atrial fibrillation, stroke, renal damage, pulmonary embolism, deep vein thrombosis, percutaneous translumenal coronary angioplasty, disseminated intravascular coagulation, sepsis, artificial organs, shunts or prostheses. In the exemplary methods of the invention, the VWF binding or/and thrombosis are associated with thrombotic thrombocytopenia, other types of microagniopathy, and VWD (type II and platelet type).

The present invention further contemplates kits comprising compositions of the present invention and a delivery device for the administration of the novel compositions described herein. In preferred embodiments, the kits comprise a delivery device that is a preloaded catheter comprising a composition of the claimed invention. The kits may further comprise a second anticoagulant agent in a suitable delivery device.

In certain embodiments, the present invention provides a method of treating or preventing thrombosis associated with sepsis or endotoxemia in a mammal comprising the step of administering an inhibitor of GPIb-IX binding to von Willebrand factor selected from the group consisting of a blocking antibody, a soluble GPIb fragment, a DNA aptamer, a peptide or a small molecule that block VWF-GPIb interaction in an amount effective for treating thrombosis, and the inhibitor comprising a coating.

In some embodiments, the blocking antibody is selected from the group consisting of a von Willebrand factor antibody, a GPIbα antibody, a GPIbβ antibody, a GPIX antibody or a GPV antibody.

In some aspects, the thrombosis is arterial thrombosis. In other aspects, the thrombosis is microvascular thrombosis. In some embodiments, the thrombosis is associated with sepsis. In some embodiments, the thrombosis is associated with endotoxemia.

In some embodiments, a composition is provided comprising a peptide having an amino acid sequence of SIRYSGHpSL (SEQ ID NO: 1), a peptide fragment of SEQ ID NO: 1 that retains a 14-3-3 binding activity, or conservative peptide variant of SEQ ID NO: 1 that retains a 14-3-3 binding activity. In further embodiments, the peptide has a myristoyl group C₁₃H₂₇CONH— at one or both of C-terminus or N-terminus of the peptide. In still further embodiments, the composition further comprises a coating.

In further embodiments, the coating is a surfactant. In some aspects of these embodiments the surfactant is a lipid. In further aspects the lipid is lecithin. In still other embodiments the coating is a micelle.

In some embodiments, the mammal suffers from thrombotic thrombocytopenia or acquired microangiopathy. In other embodiments, the mammal suffers from hemolytic uremic syndrome.

In further embodiments, the survival rate of the mammal is increased compared to a mammal not administered said inhibitor.

In other embodiments the mammal is a human.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1. Effects of a myristoylated phospho-peptide derived from the GPIbα C-terminal 14-3-3 binding site on 14-3-3 binding to GPIb-IX and ristocetin-induced platelet aggregation. (FIG. 1A) A schematic showing the platelet receptor, GPIb-IX and 14-3-3 binding sites. (FIG. 1B) Platelet lysates were incubated with recombinant 14-3-3-conjugated beads or control MBP-conjugated beads in the presence of myristoylated peptides, MPαC, and control peptides, MαC or MαCsc, or DMSO. Bead-bound GPIb-IX was immunoblotted with an anti-GPIbα monoclonal antibody. (FIG. 1C) Platelet-rich plasma (PRP) from healthy donors was preincubated with increasing concentrations of MPαC, and then stimulated with ristocetin to induce platelet aggregation. (FIG. 1D, FIG. 1E) Ristocetin-induced platelet aggregation in the presence of 100 μM MPαC, myristoylated control peptides, MαC or MαCsc, or vehicle (DMSO) (FIG. 1D) or in the presence of non-myristoylated phospho-peptide (PαC), non-phosphorylated peptide (αC) with identical sequence to MPαC, or myristic anhydride (MA) (FIG. 1E).

FIG. 2. The 14-3-3 binding peptide, MPαC, specifically inhibits GPIb-IX-dependent platelet agglutination. (FIG. 2A and FIG. 2B) PRP was preincubated with myristoylated peptides, MPαC, MαC or MαCsc, or vehicle (DMSO) together with 1 mM integrin inhibitor, RGDS. Ristocetin (1.25 mg/ml) was added to induce GPIb-IX-specific platelet agglutination. Quantitative data from 4 experiments are shown in FIG. 2B. (FIG. 2C, FIG. 2D and FIG. 2E) PRP was preincubated with MPαC, MαC or MαCsc, or DMSO, then stimulated with collagen (FIG. 2C), ADP (FIG. 2D) or thromboxane A2 analog, U46619 (FIG. 2E), to induce platelet aggregation.

FIG. 3. MPαC inhibits vWF binding to platelets. (FIG. 3A) Washed human platelets were preincubated with MPαC or control peptides MαC or MαCsc and then incubated with 1 mg/ml ristocetin in the presence (vWF) or absence (Control) of 30 μg/ml vWF. vWF binding was detected using FITC-labeled anti-vWF antibody and flow cytometry. (FIG. 3B) Quantitative data from 3 experiments. vWF binding index=Total fluorescence(geomean)/background fluorescence-1.

FIG. 4. Effects of MPαC on vWF-dependent platelet adhesion under flow and bleeding time. (FIG. 4A) Platelets were preincubated with MPαC or control peptides MαC or MαCsc. and then perfused through vWF-coated capillary tubes. Numbers of adherent platelets were counted at 10 randomly selected time frames and locations (mean±SD). (FIG. 4B) Peptides were infused into C57B mice in a double-blinded fashion. After 5 min, tail bleeding times were determined. λ, Bleeding time of individual mice. The bars represent median bleeding time of each group. Median bleeding time of MPαC-treated mice was significantly prolonged compared to control peptide-treated mice (P<0.0001). (FIG. 4C) A novel 14-3-3-dependent mechanism for regulating receptor function of GPIb-IX.

FIG. 5. Inhibition of ristocetin-induced platelet aggregation by MSDαC. Platelet-rich plasma (PRP) was preincubated with or without MSDαC peptide or with DMSO at room temperature for 5 min, and then exposed to ristocetin to induced VWF-dependent platelet aggregation. MSDαC completely inhibited ristocetin-induced platelet aggregation, indicating that this peptide has similar effects as MPαC peptide in inhibiting VWF-induced platelet aggregation.

FIG. 6 depicts results showing that micellar MPαC inhibits ristocetin-induced platelet agglutination.

FIG. 7 shows the effect of MPαC on arterial thrombosis in a FeCl₃-induced mouse carotid artery thrombosis model.

FIG. 8 depicts the therapeutic effect of MPαC on LPS-induced reduction in platelet counts.

FIG. 9 depicts Mallory's phophotungstic acid hematoxylin method (PTAH) stained kidney sections from ADAMTS13 knockout mice showing platelet and fibrin rich thrombi (dark color) in glomeruli induced by LPS (middle panels) which is inhibited by MPαC treatment (lower panels).

FIG. 10 demonstrates an improved survival rate in a mouse model of LPS-induced sepsis as a result of micellar MPαC injection.

FIG. 11 depicts the effect of micellar MPαC on platelet adhesion to histamine-treated endothelial cells under shear stress.

### DESCRIPTION OF THE PREFERRED EMBODIMENTS

Platelet adhesion is essential for thrombosis and hemostasis. While there are a number of anti-thrombotic therapies presently being used, there remains a need for other anti-thrombotic agents.

Platelet adhesion is dependent on the binding of von Willebrand factor (vWF) to its platelet receptor, the glycoprotein (GP) Ib-IX-V complex (GPIb-IX). In the present invention it has been discovered that cell-permeable peptides, and more particularly, phospho-peptides, corresponding to the 14-3-3 binding site of GPIbαinhibit vWF binding to platelets and vWF-mediated platelet adhesion. In addition, the data described herein demonstrate that such peptides also inhibit vWF-dependent platelet agglutination induced by ristocetin or botrocetin. Furthermore, intravenous injection of the peptide compositions causes markedly prolonged bleeding time in mice, indicating a inhibition of hemostatic thrombus formation. Thus, 14-3-3 interaction with GPIbαplays critical roles in vWF binding function of GPIb-IX and in GPIb-IX-dependent platelet adhesion and thrombus formation. These results also suggest a new type of anti-platelet drugs that may potentially be useful in treating thrombotic disorders.

Importantly, the present invention provides inhibitors of GPIb-IX function as inhibitors of 14-3-3-GPIb interaction, and are effective in preventing arterial thrombosis, as well as in treating or preventing microvascular thrombosis associated with thrombotic thrombocytopenic purpura (TTP) and endotoxemia (in sepsis). In another aspect, inhibitors of GPIb-IX provided function to decrease mortality caused by endotoxemia in a mouse sepsis model.

Methods and compositions for exploiting these findings are described in further detail herein below.

### Inhibitors of GPIb-IX

### GPIBα-Derived Peptides

As discussed herein throughout and exemplified in the Examples of the present application, peptides derived from the C-terminal domain of GPIbα that retain the ability to bind the intracellular signaling molecule, 14-3-3, inhibit the interaction between 14-3-3 and GPIb-IX, and inhibit the vWF binding to platelets. Moreover, these peptides have a substantial inhibitory effect on GPIb-IX-dependent platelet aggregation. As such, these peptides will be useful in various anti-thrombotic applications including therapies designed for the treatment of pathologies or treatments such as myocardial infarction, unstable angina, atrial fibrillation, stroke, renal damage, percutaneous translumenal coronary angioplasty, disseminated intravascular coagulation, sepsis, pulmonary embolism, deep vein thrombosis, artificial organs implants, shunts implants and prostheses such as artificial heart valves and the like. Indeed, it is contemplated that the compositions of the present invention will be useful as therapeutic agents in a like manner to the present uses of heparin and low molecular weight heparin moieties and/or presently available anti-platelet agents. Furthermore, the compositions of the present invention will be useful in treating thrombotic thrombocytopenic purpura and other types of microangiopathy caused by spontanenous interaction between circulating VWF and platelets, and in treating endotoxemia in sepsis and treating or preventing associated microvascular thrombosis. In addition, it is contemplated that the peptides of the invention also will be useful in a variety of combination therapies. Such applications are discussed in further detail elsewhere in the specification.

The sequence of GPIbα is well known to those of skill in the art. (Lopez et al, PNAS 84, 5615-5619, 1987; Du et al., J Biol Chem 271, 7362-7367, 1996; Bodnar et al, J. Biol Chem, 274, 33474-33479, 1999). An exemplary sequence human GPIbαprotein sequence is provided at GenBank Acc. No. J02940 (reproduced herein as SEQ ID NO:3, and encoded by a polynucleotide having a nucleic acid sequence of SEQ ID NO:2). The sequence of mature Sequence of mature GPIbα protein is given in SEQ ID NO:4.

While in preferred embodiments, the sequence used herein is derived from human GPIbα, it is contemplated that the sequence also may be derived from another mammalian source such as e.g., mouse (see e.g., mouse GPIbα protein sequence at GenBank Acc. No. NM_{—}010326 for murine protein and nucleic acid sequences). Other sequences for GPIbαproteins are known to those of skill in the art. For example, additional compositions that contain GPIbα those of skill in the art are referred to e.g., U.S. Pat. No. 6,177,059 (incorporated herein by reference in its entirety), which teaches compositions of GPIb as lipid conjugates).

In the present application, a peptide derived from the C-terminal residues 602-610 of GPIbα was prepared and shown to inhibit 14-3-3 interaction with GPIb-IX and to have beneficial inhibitory properties on VWF binding, platelet aggregation and to significantly prolong the time of occlusive arterial thrombosis formation, while moderately prolonging bleeding time in an in vivo model. This peptide comprises residues SIRYSGHSL (SEQ ID NO:1). In specific embodiments, it is contemplated that the serine residue derived from S⁶⁰⁹ in the fragment is phosphorylated. Thus, the sequence of the peptide is SIRYSGHpS⁶⁰⁹L (low case p indicate phosphorylation).

In particularly preferred embodiments, the peptide is derivatized at the C- or N-terminus with a fatty acyl group. In preferred embodiments, the fatty acyl moiety is a myristoyl moiety, however, it is contemplated that other saturated or unsaturated fatty acyl moieties from C2 to C24 may be used as the fatty acyl moieties.

Other preferred peptides produced in the present invention include, but are not limited to myristoylated SIRYSGHDL (SEQ ID NO:8), SIRYSGHEL (SEQ ID NO:9), RYSGHpSL (SEQ ID NO:10), or longer GPIbα cytoplasmic domain sequence containing the 14-3-3 binding site. The cytoplasmic domain of GPIbα has the following sequence:

(SEQ ID NO:11)

SWVGHVKPQALDSGQGAALTTATQTTHLELQRGRQVTVPRAWLLFLRGSL
PTFRSSLFLWVRPNGRVGPLVAGRRPSALSQGRGQDLLSTVSIRYSGHSL.

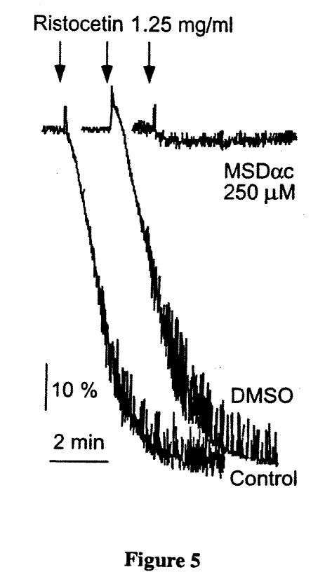

The peptides used in the present invention may be peptides of 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acid residues in length derived from the C-terminal portion of the GPIbα as long as the peptide retain the ability to bind 14-3-3. Therefore, other exemplary peptides may include, e.g., VSIRYSGHSL (SEQ ID NO: 12); TVSIRYSGHSL (SEQ ID NO: 13); STVSIRYSGHSL (SEQ ID NO: 14); LSTVSIRYSGHSL (SEQ ID NO: 15); LLSTVSIRYSGHSL (SEQ ID NO: 16); DLLSTVSIRYSGHSL (SEQ ID NO: 17); QDLLSTVSIRYSGHSL (SEQ ID NO: 18); GQDLLSTVSIRYSGHSL (SEQ ID NO:19); RGQDLLSTVSIRYSGHSL (SEQ ID NO:20); GRGQDLLSTVSIRYSGHSL (SEQ ID NO:21); QGRGQDLLSTVSIRYSGHSL (SEQ ID NO:22); SQGRGQDLLSTVSIRYSGHSL (SEQ ID NO:23); LSQGRGQDLLSTVSIRYSGHSL (SEQ ID NO:24); ALSQGRGQDLLSTVSIRYSGHSL (SEQ ID NO:25); SALSQGRGQDLLSTVSIRYSGHSL (SEQ ID NO:26); PSALSQGRGQDLLSTVSIRYSGHSL (SEQ ID NO:27). As one skilled in the art will readily recognize these peptides are derived from the C-terminus of the sequence of SEQ ID NO:4. In particularly preferred embodiments, one or more of the serine residues is substituted with an aspartic acid or glutamic acid residue in order to simulate the effect of phosphorylation of those residues. Thus, other exemplary peptides of the invention include e.g., any of sequences VSIRYSGHSL (SEQ ID NO: 12); TVSIRYSGHSL (SEQ ID NO:13); STVSIRYSGHSL (SEQ ID NO: 14); LSTVSIRYSGHSL (SEQ ID NO: 15); LLSTVSIRYSGHSL (SEQ ID NO: 16); DLLSTVSIRYSGHSL (SEQ ID NO: 17); QDLLSTVSIRYSGHSL (SEQ ID NO: 18); GQDLLSTVSIRYSGHSL (SEQ ID NO: 19); RGQDLLSTVSIRYSGHSL (SEQ ID NO:20); GRGQDLLSTVSIRYSGHSL (SEQ ID NO:21); QGRGQDLLSTVSIRYSGHSL (SEQ ID NO:22); SQGRGQDLLSTVSIRYSGHSL (SEQ ID NO:23); LSQGRGQDLLSTVSIRYSGHSL (SEQ ID NO:24); ALSQGRGQDLLSTVSIRYSGHSL (SEQ ID NO:25); SALSQGRGQDLLSTVSIRYSGHSL (SEQ ID NO:26); PSALSQGRGQDLLSTVSIRYSGHSL (SEQ ID NO:27) or other C-terminal fragments of a sequence of SEQ ID NO:4 in which one or more of the serine or threonine residues has been substituted with an aspartic acid or a glutamic acid residue. Such peptides are preferably myristoylated. In an exemplary embodiment, a myristoylated peptide with the sequence of C13H27CONH-SIRYSGHDL was synthesized and named “MSDαC”. The amino acid sequence of this peptide is derived from the GPIbα C-terminal SIRYSGHpSL sequence with a mutation that replaces phosphorylated serine 609 residue with an aspartic acid to simulate phosphoserine. Platelet-rich plasma (PRP) were preincubated with or without MSDαC peptide or with DMSO at room temperature for 5 min, and then exposed to ristocetin to induced VWF-dependent platelet aggregation. MSDαC completely inhibited ristocetin-induced platelet aggregation, indicating that this peptide has similar effects as MPαC peptide in inhibiting VWF-induced platelet aggregation (see FIG. 5). Similar such studies may be performed with any other C-terminal residue of the sequence of SEQ ID NO:4 as described herein.

In those embodiments in which the GPIbα-derived phospho-peptide will be delivered as a therapeutic agent, it is contemplated that the GPIbα derived, myristoylated phospho-peptides may be modified to enhance their uptake, circulation, and/or other modifications to render the peptides more therapeutically effective. Thus, it may be desirable to prevent the degradation of the peptides in order to prolong the effects thereof, and as, such prolong the effects of the GPIbα-derived peptide as an inhibitor of platelet aggregation and the like in the circulation of an individual suffering from or at risk of developing e.g., thrombosis. This may be achieved through the use of non-hydrolyzable peptide bonds, which are known in the art, along with procedures for synthesis of peptides containing such bonds. Non-hydrolyzable bonds include —[CH₂NH]— reduced amide peptide bonds, —[COCH₂]— ketomethylene peptide bonds, —[CH(CN)NH]— (cyanomethylene)amino peptide bonds, —[CH₂ CH(OH)]— hydroxyethylene peptide bonds, —[CH₂O]— peptide bonds, and —[CH₂S]— thiomethylene peptide bonds (see e.g., U.S. Pat. No. 6,172,043).

It is also contemplated that myristoylation of the peptide confers membrane interaction and/or membrane permeability to this peptide. Thus, other moiety or method that renders this peptide membrane-permeable or membrane attachable may also be used for the effectiveness of this peptide in inhibiting 14-3-3 function and the function of GPIb-IX to mediate VWF binding, platelet adhesion and activation, and thrombus formation. Example of the method that renders peptide membrane permeable include the signal peptides, carrier peptides and other lipophilic moieties.

GPIbα-derived proteins useful in the invention can be linear, or maybe circular or cyclized by natural or synthetic means. For example, disulfide bonds between cysteine residues may cyclize a peptide sequence. Bifunctional reagents can be used to provide a linkage between two or more amino acids of a peptide. Other methods for cyclization of peptides, such as those described by Anwer et al. (Int. J. Pep. Protein Res. 36:392-399, 1990) and Rivera—Baeza et al. (Neuropeptides 30:327-333, 1996) are also known in the art.

Furthermore, nonpeptide analogs of the GPIbα-derived peptides of the invention that provide a stabilized structure or lessened biodegradation, are also contemplated. Peptide mimetic analogs can be prepared based on a GPIbα peptide by replacing one or more amino acid residues of the protein of interest by nonpeptide moieties. Preferably, the nonpeptide moieties permit the peptide to retain its natural confirmation, or stabilize a preferred, e.g., bioactive confirmation and an overall positive charge. One example of methods for preparation of nonpeptide mimetic analogs from peptides is described in Nachman et al., Regul. Pept. 57:359-370 (1995). The term “peptide” as used herein embraces nonpeptide analogs, mimetics and modified peptides.

The GPIbα derived peptides used in the therapeutic methods of the present invention may be modified in order to improve their therapeutic efficacy. Such modification of therapeutic compounds may be used to decrease toxicity, increase circulatory time, or modify biodistribution. A strategy for improving drug viability is the utilization of water-soluble polymers. Various water-soluble polymers have been shown to modify biodistribution, improve the mode of cellular uptake, change the permeability through physiological barriers, and modify the rate of clearance from the body. (Greenwald et al., Crit. Rev Therap Drug Carrier Syst. 2000; 17:101-161; Kopecek et al., J Controlled Release., 74:147-158, 2001). To achieve either a targeting or sustained-release effect, water-soluble polymers have been synthesized that contain drug moieties as terminal groups, as part of the backbone, or as pendent groups on the polymer chain.

Polyethylene glycol (PEG), has been widely used as a drug carrier, given its high degree of biocompatibility and ease of modification. Harris et al., Clin Pharmacokinet. 2001; 40(7):539-51 Attachment to various drugs, proteins, and liposomes has been shown to improve residence time and decrease toxicity. (Greenwald et al., Crit. Rev Therap Drug Carrier Syst. 2000; 17:101-161; Zalipsky et al., Bioconjug Chem. 1997; 8:111-118). PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications (Nathan et al., Macromolecules. 1992; 25:4476-4484; Nathan et al., Bioconj Chem. 1993; 4:54-62).

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate.

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the GPIbα derived proteins described herein for therapeutic delivery.

### Blocking Antibodies

Anti-GPIb-IX antibodies or Anti-VWF antibodies are commercially available and known to those of skill in the art. Antibodies that are contemplated for use are generally described in U.S. Pat. No. 5,486,361, hereby incorporated by reference in its entirety. Anti-GPIb-IX antibodies that may block VWF binding include but are not limited to AN51 (Ruan et al, Br J Hematol, 1981 December; 49(4):511-9), 6D1 (Coller et al, Blood 1983 January;61(1):99-110), 6B4 (Thromb Haemost 2006; 96:671-84), AK2 (Berndt et al, Biochemistry 1988 Jan. 26; 27(2):633-40), API (Kunicki, T. J., R. R. Montgomery, and D. Pidard. 1983. Blood. 62(Suppl):260a), Weiss HJ and Sussman Blood. 1986 July; 68(1):149-56). Anti-GPIb antibodies are also commercially available (BD, Sigma, Signal transduction).

### Soluble GPIb-IX and VWF Fragments

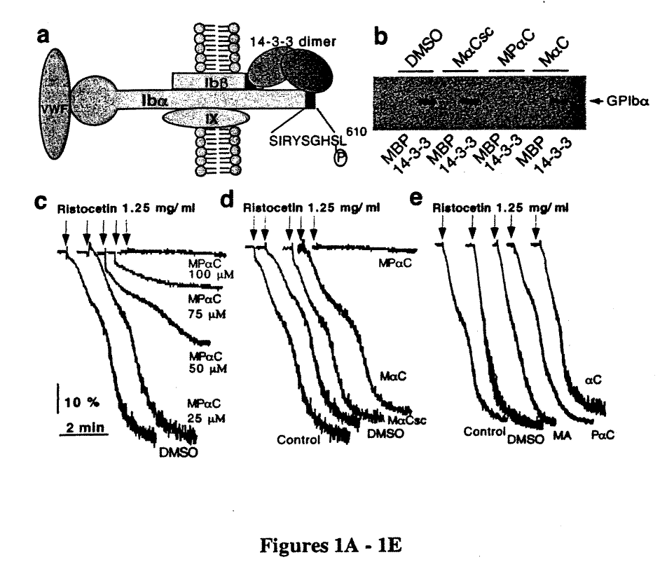

Fragments of GPIbα, GPIbβ, GPIX and GPV are also contemplated by the present invention. In general, fragments of GPIbα and GPIbβ should retain the ability to bind 14-3-3. As discussed previously herein, phosphorylation-dependent binding sites for the dimeric 14-3-3 are present in the cytoplasmic domains of both GPIbα and GPIbβ (FIG. 1A). A binding site in GPIbα resides in the C-terminal SIRYSGHpS⁶⁰⁹L (SEQ ID NO:1) sequence in which Ser⁶⁰⁹ is constitutively phosphorylated in resting platelets (Bodnar et al., J. Biol. Chem., 274:33474-33479 (1999)). The binding site in GPIbβ is located in the RLpS¹⁶⁶LTDP sequence (Andrews et al., Biochemistry, 37:638-647 (1998); Calverley et al., Blood, 91:1295-1303 (1998)) in which Ser¹⁶⁶ can be phosphorylated by cAMP-dependent protein kinase (PKA) upon activation by elevated intracellular cAMP (Wardell et al., J. Biol. Chem., 264:15656-15661 (1989)).

One of ordinary skill in the art can produce fragments of GPIbα and GPIbβ that comprise the 14-3-3 binding site, and therefore these fragments are specifically contemplated for use according to the materials and methods of the present invention. In addition, soluble fragments of VWF are also contemplated by the present invention. The amino acid sequence within VWF that is responsible for binding to GPIb has been elucidated (Matsushita et al., 2000, The Journal of Biological Chemistry 275(15): 11044-11049). Therefore, it is well within the skill of one with knowledge in the relevant art to able make and use soluble fragments of VWF comprising the binding site that block its ability to bind to GPIb.

### DNA Aptamers

Methods of identifying and recovering aptamers directed against a biological target are generally disclosed in U.S. Pat. No. 5,840,867 and U.S. Pat. No. 5,582,981, each of which is incorporated herein by reference in its entirety. These methods are known to one of ordinary skill in the art. Also contemplated are aptamers directed against VWF, which are disclosed in U.S. application 20060264369, incorporated herein by reference in its entirety.

The present invention contemplates aptamers that bind to GPIbα, GPIbβ, GPIX, GPV, VWF or 14-3-3 protein targets. According to the methods disclosed herein, binding of the aptamer to the target inhibits GPIb-IX binding to VWF. Suitable aptamers according to the present invention can be determined by those of ordinary skill in the art.

Peptides or Small Molecule Compounds that Block VWF-GPIb-IX Interaction

Peptides in addition to those disclosed herein are also contemplated for use in the present invention. Compounds that can inhibit the VWF-GPIb-IX interaction are generally disclosed in U.S. Pat. No. 7,235,558 and U.S. Patent Application 20070173489, which are each incorporated herein by reference in their entirety. In addition, mimotopes and anti-mimotopes of human platelet glycoprotein Ib/IX are disclosed in U.S. Pat. No. 5,877,155, which is incorporated herein by reference in its entirety. In certain embodiments, modified peptides as discussed herein are also contemplated for use.

### Methods of Making and Isolating GPIbα Derived Peptides

The present invention provides GPIbα-based proteins and peptides either as medicaments themselves, or for use in combinations with other hemostatic agents. Such GPIbα peptides may be produced by conventional automated peptide synthesis methods or by recombinant expression. General principles for designing and making proteins are well known to those of skill in the art.

A. Peptide Synthesis

The preferred method for making the peptides used in the present invention is in solution or on a solid support in accordance with conventional fmoc-based techniques. The peptides can be prepared from a variety of synthetic or enzymatic schemes, which are well known in the art. Where short peptides are desired, such peptides are prepared using automated peptide synthesis in solution or on a solid support in accordance with conventional techniques. Various automatic synthesizers are commercially available and are used in accordance with known protocols. See, for example, Stewart and Young, Solid Phase Peptide Synthesis, 2d. ed., Pierce Chemical Co., (1984); Tam et al., J. Am. Chem. Soc., 105:6442, (1983); Merrifield, Science, 232: 341-347, (1986); and Barany and Merrifield, The Peptides, Gross and Meienhofer, eds, Academic Press, New York, 1-284, (1979); Fields, (1997) Solid-Phase Peptide Synthesis. Academic Press, San Diego.); Andersson et al., Large-scale synthesis of peptides. Biopolymers (Pept. Sci.), 55, 227-250 (2000); Burgess et al., DiSSiMiL: Diverse Small Size Mini-Libraries applied to simple and rapid epitope mapping of a monoclonal antibody. J. Pept. Res., 57, 68-76, (2001); Peptides for the New Millennium, Fields, J. P. Tam & G. Barany (Eds.), Kluwer Academic Publisher, Dordrecht. Numerous other documents teaching solid phase synthesis of peptides are known to those of skill in the art and may be used to synthesis epitope arrays from any allergen.

For example, the peptides are synthesized by solid-phase technology employing an exemplary peptide synthesizer such as a Model 433A from Applied Biosystems Inc. This instrument combines the FMOC chemistry with the HBTU activation to perform solid-phase peptide synthesis. Synthesis starts with the C-terminal amino acid. Amino acids are then added one at a time till the N-terminus is reached. Three steps are repeated each time an amino acid is added. Initially, there is deprotection of the N-terminal amino acid of the peptide bound to the resin. The second step involves activation and addition of the next amino acid and the third step involves deprotection of the new N-terminal amino acid. In between each step there are washing steps. This type of synthesizer is capable of monitoring the deprotection and coupling steps.

At the end of the synthesis the protected peptide and the resin are collected, the peptide is then cleaved from the resin and the side-chain protection groups are removed from the peptide. Both the cleavage and deprotection reactions are typically carried out in the presence of 90% TFA, 5% thioanisole and 2.5% ethanedithiol. After the peptide is separated from the resin, e.g., by filtration through glass wool, the peptide is precipitated in the presence of MTBE (methyl t-butyl ether). Diethyl ether is used in the case of very hydrophobic peptides. The peptide is then washed a plurality of times with MTBE in order to remove the protection groups and to neutralize any leftover acidity. The purity of the peptide is further monitored by mass spectrometry and in some case by amino acid analysis and sequencing.

The peptides also may be modified, and such modifications may be carried out on the synthesizer with very minor interventions. An amide could be added at the C-terminus of the peptide. An acetyl group could be added to the N-terminus. Biotin, stearate and other modifications could also be added to the N-terminus.

The purity of any given peptide, generated through automated peptide synthesis or through recombinant methods, is typically determined using reverse phase HPLC analysis. Chemical authenticity of each peptide is established by any method well known to those of skill in the art. In certain embodiments, the authenticity is established by mass spectrometry. Additionally, the peptides also are quantified using amino acid analysis in which microwave hydrolyses are conducted. In one aspect, such analyses use a microwave oven such as the CEM Corporation's MDS 2000 microwave oven. The peptide (approximately 2 μg protein) is contacted with e.g., 6 N HCl (Pierce Constant Boiling e.g., about 4 ml) with approximately 0.5% (volume to volume) phenol (Mallinckrodt). Prior to the hydrolysis, the samples are alternately evacuated and flushed with N₂. The protein hydrolysis is conducted using a two-stage process. During the first stage, the peptides are subjected to a reaction temperature of about 100° C. and held that temperature for 1 minute. Immediately after this step, the temperature is increased to 150° C. and held at that temperature for about 25 minutes. After cooling, the samples are dried and amino acid from the hydrolysed peptides samples are derivatized using 6-aminoquinolyl-N-hydroxysuccinimidyl carbamate to yield stable ureas that fluoresce at 395 nm (Waters AccQ Tag Chemistry Package). In certain aspects, the samples are analyzed by reverse phase HPLC and quantification is achieved using an enhanced integrator.

In preferred embodiments, the peptides of the present invention are made using FMOC solid-phase synthetic methods such as those described above. However, in certain embodiments, it is contemplated that those skilled in the art also may employ recombinant techniques for the expression of the proteins wherein a nucleotide sequence which encodes a peptide of the invention is inserted into an expression vector, transformed or transfected into an appropriate host cell and cultivated under conditions suitable for expression as described herein below. Recombinant methods are especially preferred for producing longer polypeptides that comprise peptide sequences of the invention. For example, see U.S. Pat. No. 5,298,293 for general methods of producing GPIbα proteins via recombinant methods, e.g., by culturing prokaryotic and eukaryotic cells transformed by a vector for the expression of human GPIbα. Those of skill in the art are particularly referred to U.S. Pat. No. 5,340,727, which describes DNA expression vectors encoding a peptide which encodes the amino acid sequence of the amino terminal region of platelet membrane glycoprotein Ibα; as well as mammalian host cells transformed by said vectors; and a process for producing a peptides from the same. The methods disclosed therein will be useful in the recombinant production of C-terminal peptides that comprise a sequence of SEQ ID NO:1 as part or all of the peptides for the purposes of the present invention.

B. Protein Purification

It will be desirable to purify the peptides of the present invention. Protein purification techniques are well known to those of skill in the art. These techniques involve, at one level, the crude fractionation of the cellular milieu to polypeptide and non-polypeptide fractions. Having separated the peptides or polypeptides of the invention from other proteins, the polypeptides or peptides of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Particularly efficient methods of purifying peptides include fast protein liquid chromatography (FPLC) and high performance liquid chromatography (HPLC).

Certain aspects of the present invention concern the purification, and in particular embodiments, the substantial purification, of an encoded polypeptide, protein or peptide. The term “purified polypeptide, protein or peptide” as used herein, is intended to refer to a composition, isolated from other components, wherein the polypeptide, protein or peptide is purified to any degree relative to its naturally-obtainable state. A purified polypeptide, protein or peptide therefore also refers to a polypeptide, protein or peptide, free from the environment in which it may naturally occur.

Generally, “purified” will refer to a polypeptide, protein or peptide composition that has been subjected to fractionation to remove various other components, and which composition substantially retains its expressed biological activity. Where the term “substantially purified” is used, this designation will refer to a composition in which the polypeptide, protein or peptide forms the major component of the composition, such as constituting about 50%, about 60%, about 70%, about 80%, about 90%, about 95% or more of the proteins in the composition.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified polypeptide, protein or peptide.

### Disorders to be Treated

GPIb-IX, as a major platelet adhesion receptor, is an excellent target for anti-thrombosis drug development. Due to the critical roles GPIb-IX plays in platelet adhesion under high shear rate flow conditions, GPIb-IX-specific inhibitors are likely to have selective effects for arterial thrombosis (for example, in stenotic arteries) or micro- or microvascular thrombosis (for example, in arterioles and capillaries). In addition, in patients suffering from thrombotic thrombocytopenic purpura and other types of thrombotic microangiopathy, micro-thrombosis can be directly induced by the spontaneous interaction between circulating vWF and GPIb-IX (Moake, Annu. Rev. Med., 53:75-88 (2002)). Thus, development of a GPIb-IX-specific anti-platelet drug will be useful in treating these types of thrombotic diseases.

It is contemplated that the compositions of the present invention will be used in the treatment of a variety of disorders in which there is a need to prevent or treat thrombosis and subsequent decrease or loss of blood flow. The examples of thromobotic disorders include but not limited to atherosclerosis, myocardial infarction, stroke, and kidney ischemia, and thrombosis in any part of the mammalian body. The composition of the present invention will also be used in the prevention and treatment of microangiopathy in which formation of microthrombi or VWF binding to platelets causes excessive consumption of platelets and/or VWF leading to subsequent bleeding diathesis. Examples of latter disorders include but not limited to thrombotic thrombocytopenic purpura, type II and platelet type von Willebrand disease (VWD).

Compositions comprising GPIb-IX inhibitors are also contemplated for use to treat or prevent hemolytic uremic syndrome. Hemolytic uremic syndrome (HUS) is a disease primarily of infancy and early childhood. It is characterized by the triad of microangiopathic hemolytic anemia, thrombocytopenia, and acute renal failure. Diarrhea and upper respiratory infection are the most common precipitating factors. HUS is the most common cause of acute renal failure in children.

HUS and thrombotic thrombocytopenic purpura (TTP) represent different ends of what is probably the same disease continuum. Endothelial cell injury appears to be the primary event in the pathogenesis of these disorders. The endothelial damage triggers a cascade of events that result in microvascular lesions with platelet-fibrin hyaline microthrombi that occlude arterioles and capillaries. The platelet aggregation results in a consumptive thrombocytopenia. The endothelial damage may result from toxins released by bacteria or viruses. In TTP, the hyaline microthrombi occur throughout the microcirculation, and microvascular thromboses may be found in the brain, skin, intestines, skeletal muscle, pancreas, spleen, adrenals, and heart. On the other hand, in HUS, microthrombi are essentially confined to the kidneys. Many of the infectious agents and drugs implicated in HUS/TTP are toxic to the vascular endothelium.

One of the common diseases associated with microvascular thrombosis and disseminated intravascular coagulation (DIC) is sepsis, in which entry of bacteria endotoxin into blood circulation causes severe systemic reaction and death. In sepsis, platelet-rich microvascular thrombosis in vital organs (such as lung and kidney), consumptive thrombocytopenia and disseminated intravascular coagulation (DIC) caused by endotoxemia are associated with poor prognosis and mortality. In addition, bacteria toxin-induced microvascular thrombosis is particularly manifested in patients suffering from thrombotic thrombocytopenic purpura (TTP) or hemolytic uremic syndrome (HUS). In TTP, genetic deficiency in VWF cleaving enzyme, ADAMTS13, causes release of ultra-large VWF multimers that can spontaneously bind to GPIb-IX and induce GPIb-IX-dependent microvascular thrombosis and consumptive thrombocytopenia, which is often induced by bacteria toxins. Thus, ADAMTS13-deficiency may serve as a useful model for studying the bacteria toxin-induced microvascular thrombosis. It has been unclear whether and how Glb-IX plays a role in bacteria endotoxin-induced microvascular thrombosis, thrombocytopenia and mortality.

The peptides of the present invention, e.g., peptides comprising SEQ ID NO: 1 or conservative variants thereof, inhibit VWF-dependent platelet adhesion and aggregation. The peptides have been shown to be useful in prolonging the time of occlusive arterial thrombus formation in a FeCl₃-induced arterial thrombosis model in a mammal and as such, will be useful as anti-thrombotic agents both in therapeutic and prophylactic methods. As such, these peptides will be useful as anti-thrombotic agents and/or anti-platelet agents.

As explained herein throughout there are at least two types of medicaments that may arise from the present invention. Firstly, the present invention provides compositions that comprise GPIbα fragments described herein above as anti-thrombotic agents alone. Alternatively, the peptides and/or inhibitors described herein may be combined with other therapeutic agents for the treatment of thrombosis and other disorders of the cardiovascular circulatory system that require and increase in the flow or reducing blockage of the vessels.

The peptide and/or inhibitors in this invention may also be used to block 14-3-3 interaction with other ligands of 14-3-3 that are present in all eukaryotic cells and are potentially important in other cellular functions including but not limiting to cell protection against apoptosis, cell proliferation, cell cycle and intracellular signal transduction as described in the literature (Fu et al., Annu Rev Pharmacol Toxicol 40, 617-647, 2000).

The present invention therefore contemplates a method of treating or preventing thrombosis in a mammal suffering from sepsis comprising the step of administering an inhibitor of GPIb-IX binding to von Willebrand factor selected from the group consisting of anti-GPIb-IX antibodies, soluble GPIb-IX fragments, DNA aptamers, and peptides or small molecule compounds that block VWF-GPIb-IX interaction in an amount effective for treating thrombosis, said inhibitor comprising a coating.

In some embodiments, GPIb-IX inhibitors are also contemplated for use in treating or preventing sepsis-induced microvascular thrombosis and thrombocytopenia in a patient. It is contemplated that use of the compositions of the present invention for treating or preventing the disorders disclosed herein will result in an increase in survival in those patients receiving said compositions, relative to those patients not receiving said compositions. In some embodiments, the increase in survival rate is contemplated to be about 5%. In other embodiments, the increase is about 6%, or about 7%, or about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or about 31%, or about 32%, or about 33%, or about 34%, or about 35%, or about 36%, or about 37%, or about 38%, or about 39%, or about 40%, or about 41%, or about 42%, or about 43%, or about 44%, or about 45%, or about 46%, or about 47%, or about 48%, or about 49%, or about 50%, or about 51%, or about 52%, or about 53%, or about 54%, or about 55%, or about 56%, or about 57%, or about 58%, or about 59%, or about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% or more.

A. Treatment using GPIbα Fragments

GPIbα fragments described herein have an anti-platelet activity. Therefore, the fragments or combinations will be useful for the treatment of any disorder that is presently treated using anticoagulant therapy, such as by the use of heparin-based medicaments or other antiplatelet agents such as e.g., Aggrastat™, Aggrenox™, Agrylin™, Flolan™, Integfilin™, Presantine™, Plavix™, Pletal™, REoPrO™, Coumdin, Fragmin™, Hep-Lock™, Lovenox™, Miradon™ and the like. Such disorders include pulmonary embolism, unstable angina, myocardial infarction, deep vein thrombosis, atrial fibrillation with embolization, acute and chronic coagulopathies (disseminated intravascular coagulation), for prevention of clotting in arterial and cardiac surgery, for prophylaxis and treatment of peripheral arterial embolism. The GPIbα-derived peptide described in the present invention will be also be used to treat or prevent thrombotic thrombocytopic purpura, other types of microangiopathy that are mediated by interaction between VWF and platelets, platelet type or type IIb von Willebrand diseases in which there is an increased binding of VWF to platelets (either caused by a defect in GPIb or in VWF), and microvascular thrombosis associated with sepsis or entering of bacterial toxin into the circulation as well as consequent organ damage (e.g., in lung, kidney, liver). The compositions described herein may be useful as anti-platelet agents in blood transfusions, extracorporeal circulation, dialysis procedures as well as blood sampling for laboratory procedures. The compositions also may be used to maintain the patency of an indwelling venipucture device that is being used for intermittent injection or infusion therapy or blood sampling. The compositions may be particularly useful in surgical procedures to prevent the formation of blood clots. Such indications are particularly desirable for patients undergoing abdominal surgery to reduce the risk of thromboembolic complications, patients undergoing knee or hip replacement therapy during and following the replacement procedure, as well as a general prophylactic to prevent clot formation at a later stage. The compositions also may be useful in the treatment of subjects that are under risk of thromboembolic complications due to severely restricted mobility e.g., during acute illness. Any such disorders may be readily treated by the compositions described herein. The therapeutic methods include both medical therapeutic and/or prophylactic administration, as appropriate.

As used herein, the term “inhibits platelet aggregation” includes its generally accepted meaning which includes prohibiting, slowing, or reducing the severity or degree of platelet aggregation. Such an inhibition may be measured as a function of time taken for a given platelet sample to aggregate. Aggregation can be determined using a turbidometric platelet aggregometer. Methods of determining the efficacy of the agents include coagulation testing, monitoring the time of bleeding, FeCl₃-induced carotid artery thrombosis analysis, and determining hemoglobin levels of an animal.

For example, clots may be analyzed in vitro in an assay in which citrated plasma (e.g., 1100 μl) is mixed with 15 pt of radiolabelled human fibrinogen (e.g., about 40,000 cpm/clot). plasma (35 μl) is mixed with 35 μl of Tris-buffered saline (TBS) containing 10 mM CaCl2 and thrombin (1 U/ml) in twelve 65-mm plastic tubes and clotted for 1 hour at 37° C. The clots are washed in TBS, the supernatant is removed, and then 100 pt of TBS or 25 μg of purified plasminogen activator fragment is added to tubes in duplicate. Clot lysis is initiated by adding 0.1 U of plasminogen activator per tube. The clots are incubated at 37° C. for 5 hours and the amount of lysis was determined by sampling for the release of radiolabeled fibrin degradation products into the supernatant, as described (Reed, G. L. III et al., Proc. Natl. Acad. Sci. USA 87:1114-1118 (1990)).

Experimental models of pulmonary embolism are known to those of skill in the art. In such a model a small mammal, e.g., a rat is an anesthetized by intramuscular injection (0.4 ml) of a mixture of ketamine and acepromazine (two parts acepromazine [10 mg/ml] to one part ketamine [100 mg/ml]). Intraperitoneal injections are repeated as necessary to keep the animals anesthetized. After an anterior midline incision is made in the neck, the jugular vein and the carotid artery are exposed by blunt dissection and cannulated with 20G catheters that are secured at the proximal and distal ends with 4-0 silk sutures. The catheters are capped with three-way stopcocks.

Citrated human plasma is mixed with 125I-fibrinogen to achieve about 1,000,000 cpm/ml. Individual clots are formed by mixing 125I-fibrinogen-labeled plasma (45 μl) with 2.5 μl of bovine thrombin (100 U/ml) and 2.5 pt of calcium chloride (0.4 M). These clots are incubated at 37° C. for 90 minutes, compressed, and washed thoroughly with saline three times to remove unbound proteins. The radioactive content of the clots is measured in a gamma counter immediately before clot injection. Blood samples are drawn at base line and at the end of the experiment. Sodium iodide (10 mg) is injected to block thyroid uptake. Clots are embolized into the lungs by injection through the internal jugular vein. Animals weighing less than 1 kg received three clots; those weighing 1 kg or more received four clots. Successful embolization is evidenced by the accumulation of radioactivity in the thorax. After the clots are injected, the animals are turned on their sides to ease breathing.

All animals receive weight-adjusted heparin at 100 U/kg (bolus), a dose sufficient to keep the activated partial thromboplastin time (aPTT) above 150 seconds throughout the procedure. The anti-platelet agent being tested is administered intravenously as a single dose (e.g., 20 mg/kg). The plasminogen activator is given as a continuous infusion over 2 hours (1 or 2 mg/kg in 5 ml normal saline). Animals are observed for a total of four hours after pulmonary embolization and then killed by lethal injection of anesthesia or by CO₂ inhalation. The thorax is dissected and all intrathoracic structures are removed for gamma counting to detect residual thrombi. The percentage of clot lysis was determined for each animal by dividing the total residual radioactivity in the thorax (cpm) by that in the initial thrombi. In addition to the above methods, specific examples of assays for determining platelet adhesion, animal bleeding times and platelet aggregation are provided in Example 1.

Of course, it should be understood that the GPIbα fragments may form part of a therapeutic regimen in which the GPIbα-based peptide treatment is used in combination with a plurality of other therapies for the given disorder. As such, combination therapy is specifically contemplated. In combination therapy, the GPIbα-based peptide composition is administered with another anticoagulant or anti-platelet agent. Such agents are well known to those of skill in the art and include, but are not limited to Aggrastat™, Aggrenox™, Agrylin™, Flolan™, Integrilin™, Presantine™, Plavix™, Pletal™, REoPrO™, Coumdin, Fragmin™, Hep-Lock™, Lovenox™, Miradon™, tinzaparin, certoparin, parnaparin, nadroparin, ardeparin, enoxaparin, reviparin, reviparin, dalteparin, and fraxiparin. In specific embodiments, the one or more of the compositions may be provided in a catheter.

From the above discussion, it should be understood that the disorder that may be treated by the compositions of the present invention are limited only by the fact that the disorder needs a therapeutic intervention which inhibits platelet aggregation. The doses of the agent may be modified for each individual subject. For particular guidance on the routes of administration, and uses those of skill in the art are referred to the Physician's Desk Reference for generalized descriptions of formulations, routes of administration and patient monitoring used for agents such as Aggrastat™ (see e.g., entry at pages 1933-1937, PDR, 57^{th} Edn., 2003), Aggrenox™ (see e.g., entry at pages 1023-1026, PDR, 57^{th} Edn., 2003), Agrylin™ (see e.g., entry at pages 3142-3143, PDR, 57^{th} Edn., 2003), Flolan™ (see e.g., entry at pages 1516-1521, PDR, 57^{th} Edn., 2003), Integrilin™ (see e.g., entry at pages 2138-2142, PDR, 57^{th} Edn., 2003), Presantine™ (see e.g., entry at pages 1052-2053, PDR, 57^{th} Edn., 2003), Plavix™ (see e.g., entry at pages 1098-1101, PDR, 57^{th} Edn., 2003), Pletal™ (see e.g., entry at pages 2780-2782, PDR, 57^{th} Edn., 2003), REoPrO™ (see e.g., entry at pages 1866-1870, PDR, 57^{th} Edn., 2003), Coumdin™ (see e.g., entry at pages 1074-1079, PDR, 57^{th} Edn., 2003), Fragmin™ (see e.g., entry at pages 2750-2754, PDR, 57^{th} Edn., 2003), Hep-Lock™ (see e.g., entry at pages 1284-1288, PDR, 57^{th} Edn., 2003), Lovenox™ (see e.g., entry at pages 739-744, PDR, 57^{th} Edn., 2003), Miradon™ (see e.g., entry at pages 3051-3052, PDR, 57^{th} Edn., 2003). These entries in the PDR are provided to show the level of skill in the art relating to formulating and using compositions as anticoagulants and anti-platelet agents.

Specific amounts and route of GPIbα-based anti-platelet agent administered may vary, and will be determined in the clinical trial of these agents. However, it is contemplated that those skilled in the art may administer ˜10 nmol/g body weight of the above described agents to mice via intraveneous route to achieve prolonged bleeding time.

### Pharmaceutical Compositions

Pharmaceutical compositions for administration according to the present invention can comprise either fragments of GPIbα alone as described above or in combination with other anticoagulants or antiplatelet agents. Pharmaceutical compositions comprising an inhibitor of GPIb-IX are also contemplated by the present invention. These compositions according to the present invention can comprise a single inhibitor as described herein or a combination thereof. Regardless of whether the active component of the pharmaceutical composition is a GPIbα fragment alone, a GPIbα fragment in combination with another active agent of interest, each of these preparations is in some aspects provided in a pharmaceutically acceptable form optionally combined with a pharmaceutically acceptable carrier. These compositions are administered by any methods that achieve their intended purposes. Individualized amounts and regimens for the administration of the compositions for the treatment of the given disorder are determined readily by those with ordinary skill in the art using assays that are used for the diagnosis of the disorder and determining the level of effect a given therapeutic intervention produces.

It is understood that the suitable dose of a composition according to the present invention will depend upon the age, health and weight of the recipient, kind of concurrent treatment, if any, frequency of treatment, and the nature of the effect desired. However, the dosage is tailored to the individual subject, as is understood and determinable by one of skill in the art, without undue experimentation. This typically involves adjustment of a standard dose, e.g., reduction of the dose if the patient has a low body weight.

The total dose of therapeutic agent may be administered in multiple doses or in a single dose. In certain embodiments, the compositions are administered alone, in other embodiments the compositions are administered in conjunction with other therapeutics directed to the disease or directed to other symptoms thereof.

In some aspects, the compositions of the invention are formulated into suitable pharmaceutical compositions, i.e., in a form appropriate for in vivo applications in the therapeutic intervention of a given disease. Generally, this will entail preparing compositions that are essentially free of pyrogens, as well as other impurities that could be harmful to humans or animals. In some aspects, the compositions are prepared for administration directly to the lung. These formulations are for oral administration via an inhalant, however, other routes of administration are contemplated (e.g. injection and the like). An inhaler device is any device useful in the administration of the inhalable medicament. Examples of inhaler devices include nebulizers, metered dose inhalers, dry powder inhalers, intermittent positive pressure breathing apparatuses, humidifiers, bubble environments, oxygen chambers, oxygen masks and artificial respirators. As the GPIbα fragments are relatively short peptides, such fragments may be well suited to formulation as an inhalable medicament. Therefore, it is particularly contemplated that the GPIbα fragments or the GPIbα fragments conjugated to active agents will be formulated as inhalable compositions. Further, the compositions of the invention include kits in which the inhalable medicament is formulated in a container suitable for administration via inhalation.

One will generally desire to employ appropriate salts and buffers to render the compositions stable and allow for uptake of the compositions at the target site. Generally, the pharmaceutical compositions of the invention are provided in lyophilized form to be reconstituted prior to administration. Alternatively, the pharmaceutical compositions may be formulated into tablet form or solution. Buffers and solutions for the reconstitution of the pharmaceutical compositions may be provided along with the pharmaceutical formulation to produce aqueous compositions of the present invention for administration. Such aqueous compositions will comprise an effective amount of each of the therapeutic agents being used, dissolved or dispersed in a pharmaceutically acceptable carrier or aqueous medium. Such compositions also may be useful in combination with surgical intervention to reduce the risk of blood clot development during surgery.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also are incorporated into the compositions.

Methods of formulating proteins and peptides for therapeutic administration also are known to those of skill in the art. Administration of these compositions according to the present invention will be via any common route so long as the target tissue is available via that route. Most commonly, these compositions are formulated for oral administration, such as by an inhalant. However, other conventional routes of administration, e.g., by subcutaneous, intravenous, intradermal, intramusclar, intramammary, intraperitoneal, intrathecal, intraocular, retrobulbar, intrapulmonary (e.g., term release), aerosol, sublingual, nasal, anal, vaginal, or transdermal delivery, or by surgical implantation at a particular site also is used particularly when oral administration is problematic. The treatment may consist of a single dose or a plurality of doses over a period of time.

In certain embodiments, the active compounds are prepared for administration as solutions of free base, acid or pharmacologically acceptable salts in water suitably mixed with a surfactant, such as hydroxypropylcellulose. Dispersions also are prepared in glycerol, liquid polyethylene glycols, and mixtures thereof and in oils or other solvents. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. In some aspects, the carrier is a solvent or dispersion medium containing, for example, water, ethanol, polyol (for example, glycerol, propylene glycol, and liquid polyethylene glycol, and the like), suitable mixtures thereof, and vegetable oils. The proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions is brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also are incorporated into the compositions.

In some aspects, the compositions of the present invention are formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also are derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. In certain embodiment, parenteral administration of the therapeutic compounds is carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration, a suitable dose is calculated according to body weight, body surface areas or organ size. The availability of animal models is particularly useful in facilitating a determination of appropriate dosages of a given therapeutic. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Typically, appropriate dosages are ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods of the invention are useful in fields of human medicine and veterinary medicine. Thus, the subject to be treated is a mammal, such as a human or other mammalian animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

The present invention also contemplated kits for use in the treatment of various disorders. Such kits include at least a first composition comprising the GPIbα proteins/peptides described above in a pharmaceutically acceptable carrier. In specific embodiments, the composition is provided in a catheter. Another component is a second therapeutic agent for the treatment of the disorder along with suitable container and vehicles for administrations of the therapeutic compositions. The kits may additionally comprise solutions or buffers for effecting the delivery of the first and second compositions. The kits may further comprise catheters, syringes or other delivering devices for the delivery of one or more of the compositions used in the methods of the invention. The kits may further comprise instructions containing administration protocols for the therapeutic regimens.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Synthetic peptides. Myristoylated peptides, MPαC (C₁₃H₂₇CONH-SIRYSGHpSL), MαC (C₁₃H₂₇CONH-SIRYSGHSL), MαCsc (C₁₃H₂₇CONH-LSISYGSHR), and non-myristoylated peptides, PαC (SIRYSGHpSL), and αC(SIRYSGHSL) were synthesized by standard fmoc solid-phase synthetic methods by the Protein Research Laboratory, University of Illinois at Chicago. The peptides were more than 95% in purity. Myristoylated peptides were dissolved in DMSO.

Platelet preparation and aggregation. Blood was drawn from healthy donors, and was anti-coagulated with 1/10 volume of 3.8% trisodium citrate. Platelet-rich plasma (PRP) was obtained by centrifugation at 300×g for 20 min at 22° C. Platelet aggregation was measured using a turbidometric platelet aggregometer at 37° C. with a stirring speed at 1000 rpm. To examine the effects of peptides, various concentrations of each peptide was preincubated with platelets at 22° C. for 5 min. In some experiments, intergrin inhibitor RGDS (1 mM) was also added to exclude the roles of P3 integrins.

14-3-3 binding to GPIb-IX. Binding of GPIb-IX to 14-3-3-conjugated beads were performed as previously described (Du et al., J. Biol. Chem., 271:7362-7367 (1996)). Briefly, 14-3-3-conjugated Sepharose 4B beads and maltose-binding protein (MBP)-conjugated control beads were preincubated with various concentrations of synthetic peptides at 4° C. for 1 h, and then with platelet lysates for an additional 1 h. The beads were washed three times. Bound GPIb-IX was detected by Western blot with a monoclonal antibody against GPIbα, WM23.

Flow cytometry analysis of VWF binding to platelets. Flow cytometry analysis of vWF binding to platelets has been described previously 8,9. Washed platelets (1.0×10⁷/ml) were prepared as described previously (Englund et al., J. Biol. Chem., 276:16952-16959 (2001); Bodnar et al., J. Biol. Chem. 277:47080-47087 (2002)), and resuspended in phosphate-buffered saline (PBS) solution (0.01 M NaH2PO4, 0.15 M NaCl, pH 7.4) containing 10 mM EDTA and 1% BSA. Platelets were preincubated with various concentrations of synthetic peptides at 22° C. for 10 min before incubating with purified vWF (35 μg/ml) and ristocetin (1.0 mg/ml) at 22° C. for 30 min. Platelets were washed once, further incubated in the same buffer containing 10 μg/ml FITC-labeled SZ-29 in the dark at 22° C. for 30 min and then analyzed by flow cytometry. As negative controls, platelets were incubated in the presence of ristocetin alone then incubated with FITC-labeled SZ29.

Platelet adhesion under flow. Washed platelets (3×10⁸/ml) were preincubated with various concentrations of synthetic peptides at 22° C. for 10 min, the platelets were perfused into the vWF-coated glass capillaries using a PhD syringe pump (Harvard Apparatus Inc.) at various shear rates for 8 min. Transient adhesion of platelets on the vWF-coated surface was recorded on video cassette recorder. Adherent platelets were counted in 10 randomly selected fields of 0.25 mm² within a 10 second time period.

Tail-bleeding time. Bleeding times were performed with 6-8-wk-old adult C57 black mice anesthetized with an intraperitoneal injection of avertin. The internal jugular vein was surgically exposed, and a total volume of 50 μl of a peptide (5 mM) was infused using a 27G needle. Experiments were performed in a double-blinded fashion. After 5 min, tails were amputated 5 mm from the tail tip, and immersed in a tube containing 0.15M NaCl maintained at 37° C. Bleeding was visually followed and timed. Maximum bleeding time allowed was 10 min after which the tail was cauterized. Statistical differences between the groups were examined using Mann-Whitney test and InStat software.

### Example 2

### Results and Discussion

Binding of subendothelial-bound von Willebrand factor (vWF) to its platelet receptor, glycoprotein Ib-IX (GPIb-IX), is a critical initiating step in platelet adhesion and activation (Ruggeri, Prog. Hemost. Thromb., 10:35-68 (1991); Ware, Thromb. Haemost., 79:466-478 (1998)). Despite the early belief that GPIb-IX is constitutively active in binding vWF, increasing evidence suggests that ligand binding function of GPIb-IX may be regulated by intracellular signals (Coller, Blood, 57:846-855 (1981); Englund et al., J. Biol. Chem., 276:16952-16959 (2001); Bodnar et al., J. Biol. Chem., 277:47080-47087 (2002)). It was previously shown that the cytoplasmic domain of GPIbα interacts with an intracellular signaling molecule, 14-3-3, and that a 14-3-3 binding site is located at the C-terminal domain of GPIbα (S⁶⁰²IRYSGHpSL⁶¹⁰ SEQ ID NO:1) (Du et al., J. Biol. Chem., 271:7362-7367 (1996); Bodnar et al., J. Biol. Chem., 274:33474-33479 (1999)) (FIG. 1A). To study the role of 14-3-3 in regulating GPIb-IX function, a cell-permeable myristoylated phospho-peptide named MPαC (C₁₃H₂₇CONH-SIRYSGHpSL) was prepared. This peptide was based on the sequence of 14-3-3 binding site in the C-terminal region of GPIbα with phosphorylation at the serine corresponding to Ser⁶⁰⁹. A myristoylated non-phosphorylated peptide with the identical sequence as above (MαC) and a myristoylated scrambled peptide (MαCsc) also were prepared as controls.

To examine if the above peptides interfere with 14-3-3 interaction with GPIb-IX, platelet lysates were incubated with beads conjugated with recombinant 14-3-3-maltose-binding protein (MBP) fusion protein in the absence or presence of MPαC or control peptides. GPIb-IX from platelet lysates specifically bound to 14-3-3 beads but not the control MBP-conjugated beads (FIG. 1B). The binding of GPIb-IX to 14-3-3 beads was abolished by the MPαC peptide but not the non-phosphorylated identical peptide or scrambled peptide, indicating that MPαC specifically interfere with 14-3-3 binding to GPIb-IX (FIG. 1B).

The MPαC and control peptides were then preincubated with platelet-rich plasma to determine their effect on platelet function. MPαC dose-dependently inhibited vWF-dependent platelet aggregation induced by ristocetin (a vWF modulator that mimics the effect of subendothelium to induce vWF-GPIb interaction) (FIG. 1C). In contrast, myristoylated control peptides, MαC or MαCsc, had no significant effect (FIG. 1D). Similarly, myristic anhydride (MA) also had no effect on ristocetin-induced platelet aggregation (FIG. 1E), suggesting that myristoylation is not responsible for the inhibitory effect of the peptide. On the other hand, a non-myristoylated phospho-peptide identical to MPαC (PαC) had no effect on ristocetin-induced platelet aggregation (FIG. 1E), suggesting that myristoylation is required for the inhibitory effect. Thus, these data indicate that the inhibitory effect of MPαC requires membrane permeability of this specific 14-3-3 binding peptide.

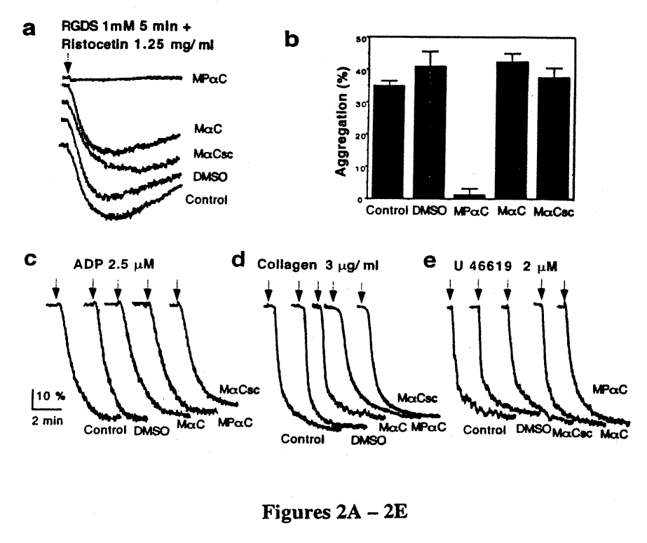

Ristocetin-induced platelet aggregation involves platelet agglutination that is caused by the cross-linking of platelets by GPIb-IX-bound vWF, platelet activation and subsequent integrin αIIbpβ3-mediated platelet aggregation. To differentiate if the inhibitory effect of MPαC result from inhibition of platelet agglutination induced by vWF binding or GPIb-IX-mediated platelet activation, the effect of MPαC on ristocetin-induced platelet agglutination was examined in the presence of RGDS peptide, which blocks integrin-dependent platelet aggregation. MPαC completely inhibited ristocetin-induced platelet agglutination in the presence of RGDS (FIG. 2A, 2B). In contrast, control peptides had no inhibitory effect. Also, MPαC inhibited botrocetin-induced platelet agglutination. Furthermore, to exclude the possibility that MPαC may affect general platelet activation process, the effect of this peptide on platelet aggregation induced by platelet agonists ADP, collagen, and thromboxane A2 analog U46619 was examined.

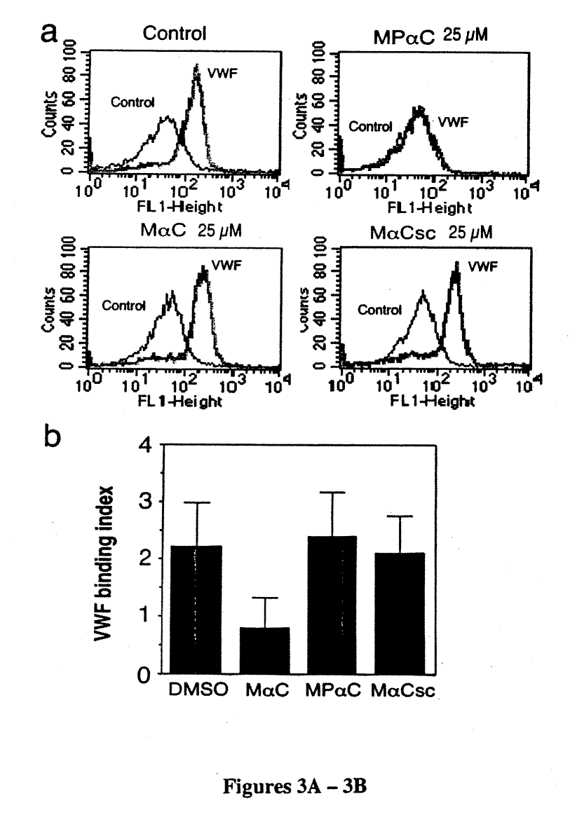

MPαC as well as control peptides had no inhibitory effect on platelet aggregation induced by these agonists (FIGS. 2C, 2D and 2E). Thus the effect of MPαC peptide is specific for GPIb-IX-dependent platelet agglutination. To directly verify if MPαC affected vWF binding, the inventors examined if MPαC affected ristocetin-induced vWF binding to GPIb-IX (to exclude the possible role of integrin in vWF binding, binding assays were performed in the presence of 10 mM EDTA or RGDS; SEQ ID NO:7). MPαC inhibited ristocetin-induced vWF binding to platelets (FIG. 3). In contrast, the control peptides had no inhibitory effect. These data indicate that a cell-permeable peptide that blocks 14-3-3 binding to GPIbα cytoplasmic domain specifically inhibits vWF binding to the extracellular ligand binding domain of GPIb-IX. Thus, 14-3-3 binding to the C-terminal region of GPIbα is required for maintaining vWF binding function of GPIb-IX in platelets.

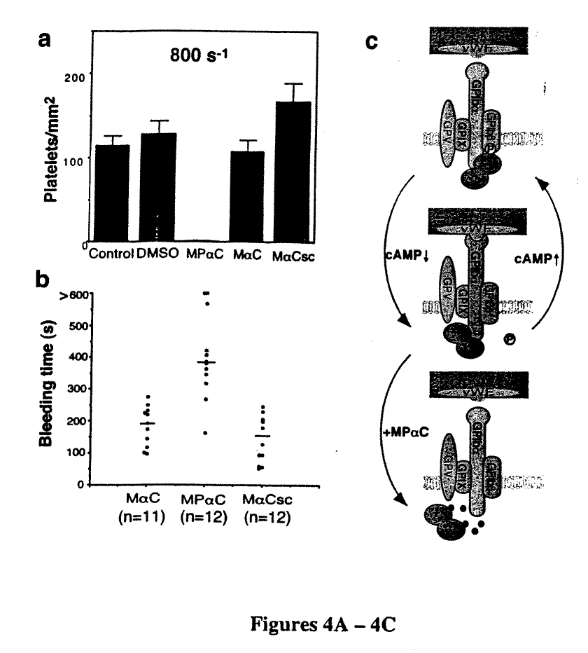

Physiological function of vWF binding to GPIb-IX is to mediate platelet adhesion under flow conditions. Thus, if 14-3-3 interaction with GPIb-IX is important for the physiological function of GPIb-IX, MPαC peptide should inhibit platelet adhesion to vWF under flow conditions. To investigate the effect of MPαC on platelets adhesion under flow, washed platelets were preincubated with peptides or vehicle (DMSO) control, then perfused into vWF-coated glass capillaries. To exclude the role of integrins, these experiments were performed in the presence of integrin inhibitor, RGDS (SEQ ID NO:7). As expected, platelets preincubated with vehicle or control peptides adhered on vWF surface. In contrast, there was almost no adhesion of platelets preincubated with MPαC (FIG. 4A). The dramatic effect of MPαC peptide to inhibit platelet adhesion not only indicates that 14-3-3 interaction with the cytoplasmic domain of GPIbα is important to platelet adhesion function, but also suggests that MPαC or similar peptides that block 14-3-3 binding to GPIbα can be used as a new class of anti-platelet agents that specifically inhibit GPIb-IX-dependent platelet adhesion.

To explore the possibility whether MPαC can be used as an anti-platelet agent in vivo, and to examine the role of 14-3-3 binding to GPIb-IX in in vivo platelet function, MPαC or control peptides MαC and MαCsc were infused into the jugular vein of anesthetized C57B mice in a double-blinded fashion. After allowing the peptides to circulate for 5 minutes, tail bleeding time analysis, which is a widely used assay for in vivo hemostatic function in mice (Offermanns et al., Nature, 389:183-186 (1997); Ware et al., Proc. Natl. Acad. Sci. USA, 97:2803-2808 (2000); Sambrano et al., Nature, 413:74-78. (2001) Li et al., Cell, 112:77-86 (2003)), were performed to these mice. Median bleeding time was significantly prolonged in mice treated with MPαC as compared with mice injected with MαC or MαCsc (p<0.0001) (FIG. 4B). These data demonstrate that 14-3-3 binding to GPIb-IX cytoplasmic domain is important for in vivo hemostatic function of platelets, and MPαC peptide indeed has anti-thrombotic effect in vivo.

The above data indicate that 14-3-3 binding to the cytoplasmic domain of GPIbα is required for GPIb-IX to maintain an active state capable of binding vWF. Phosphorylation-dependent binding sites for the dimeric 14-3-3 are present in the cytoplasmic domains of both GPIbα and GPIbβ(FIG. 1A). A binding site in GPIbα resides in the C-terminal SIRYSGHpS⁶⁰⁹L (SEQ ID NO:1) sequence in which Ser⁶⁰⁹ is constitutively phosphorylated in resting platelets (Bodnar et al., J. Biol. Chem., 274:33474-33479 (1999)). The binding site in GPIbβ is located in the RLpS¹⁶⁶LTDP sequence (Andrews et al., Biochemistry, 37:638-647 (1998); Calverley et al., Blood, 91:1295-1303 (1998)) in which Ser¹⁶⁶ can be phosphorylated by cAMP-dependent protein kinase (PKA) upon activation by elevated intracellular cAMP (Wardell et al., J. Biol. Chem., 264:15656-15661 (1989)). However, binding of 14-3-3 to GPIbα does not require cooperation of the 14-3-3 binding site in GPIbβ (Gu et al., J. Biol. Chem., 273:33465-33471 (1998)). On the other hand, deletion of the binding site in GPIbα abolishes high affinity binding of 14-3-3 to GPIb-IX, suggesting that GPIbβ alone is not sufficient to support high affinity binding of 14-3-3 5. These data suggest that GPIb-IX should normally have two different 14-3-3 interacting modes (FIG. 4C): (1) 14-3-3 dimer binds to both GPIbα and GPIβ sites when PKA is activated by elevated cAMP; and (2) 14-3-3 dimer binds only to GPIbα but not GPIbβ when cAMP level is low. PKA-mediated phosphorylation of GPIbβ inhibits vWF binding function of GPIb-IX and that dephosphorylation of GPIbβ is associated with activation of vWF binding function of GPIb-IX (Bodnar et al., J. Biol. Chem., 277:47080-47087 (2002)). Thus, the data described in this example further suggest a new model of GPIb-IX regulation. In this model, elevation of cAMP induces binding of 14-3-3 to both sites in GPIbα and GPIbβ resulting in a “resting” GPIb-IX. Decreases in cAMP level dissociate GPIbβ-14-3-3 interaction, resulting in the binding of 14-3-3 to GPIbα alone and activating vWF binding function of GPIb-IX, which can be inhibited by disrupting 14-3-3-binding to GPIbα (FIG. 4C). Therefore, 14-3-3 is a regulator of vWF binding function of GPIb-IX and is required for the activation of the receptor function of GPIb-IX.

As discussed herein above, GPIb-IX, as a major platelet adhesion receptor, is an excellent target for anti-thrombosis drug development. Due to the critical roles GPIb-IX plays in platelet adhesion under high shear rate flow conditions, GPIb-IX-specific inhibitors are likely to have selective effects for arterial thrombosis (for example, in stenotic arteries) or micro- or microvascular thrombosis (for example, in arterioles and capillaries). In addition, in patients suffering from thrombotic thrombocytopenic purpura and other types of thrombotic microangiopathy, micro-thrombosis can be directly induced by the spontaneous interaction between circulating vWF and GPIb-IX (Moake, Annu. Rev. Med., 53:75-88 (2002)). Thus, development of a GPIb-IX-specific anti-platelet drug will be useful in treating these types thrombotic diseases. Here it is demonstrated that pharmacological blockade of the interaction between 14-3-3 and GPIbα with MPαC inhibits vWF binding function of GPIb-IX and platelet adhesion, and reduces in vivo hemostatic function. Thus, MPαC or similar reagents that block GPIb-IX-14-3-3 interaction are potentially useful as a new class of anti-thrombotic agents.

### Example 3

### Additional Studies Relating to Background of the Anti-Platelet Peptides

Protein kinase A (PKA)-dependent phosphorylation of platelet glycoprotein (GP) Ibβ at Ser¹⁶⁶ negatively regulates von Willebrand factor (VWF) binding function of the glycoprotein Ib-IX complex (GPIb-IX). Thus, GPIb-IX containing a mutant GPIbβ replacing Ser¹⁶⁶ with alanine (S¹⁶⁶A) showed enhanced VWF binding when expressed in Chinese hamster ovary (CHO) cells. However, when this GPIbβ mutant was complexed with a GPIbα mutant in which Ser⁶⁰⁹, a key residue required for high affinity 14-3-3 binding, was substituted by alanine (S⁶⁰⁹A), the enhancing effect of S¹⁶⁶A mutation on vWF binding was diminished. Similarly, a PKA inhibitor, which causes GPIbβ dephosphorylation, enhanced vWF binding to wild type GPIb-IX. This effect, however, was reduced in cells expressing S⁶⁰⁹A mutant of GPIbα complexed with wild type GPIbβ, indicating that the interaction between 14-3-3 and GPIbα C-terminal domain is required for the enhancement of vWF binding induced by GPIbβ dephosphorylation. Furthermore, enhanced vWF binding function in S¹⁶⁶A cells was associated with an increased GPIb-IX dissociation from the membrane skeleton, and this effect was also reduced by S⁶⁰⁹A mutation. These data suggest a novel regulatory mechanism of GPIb-IX in which intracellular signals regulates GPIb-IX association with the membrane skeleton and ligand binding function of GPIb-IX in a 14-3-3-dependent manner. The following example experimental details and results that led to these findings.

### Materials and Methods

Reagents—Monoclonal antibody WM23 against GPIbα, monoclonal antibodies SZ29, against vWF, and SZ2, against GPIbα, and monoclonal antibody against GPIb-IX, P3, were obtained from sources known to those of skill in the art (Berndt et al., Eur. J. Biochem., 151(3):637-649 (1985); Ruan et al., Blood, 69(2):570-577 (1987); Ruan et al., Chung Hua Nei Ko Tsa Chih, 25(9):547-550, 576, (1986)). cDNA clones encoding wild type GPIbα, GPIbβ, and GPIX were obtained from sources known to those of skill in the art (Lopez et al., Proc. Natl. Acad. Sci. USA, 84(16):5615-5619 (1987); Lopez et al., Proc. Natl. Acad. Sci. USA, 85(7):2135-2139 (1988)). Bovine serum albumin (BSA), aprotinin, ristocetin, and dimethyl sulfoxide (DMSO) were purchased from Sigma (St. Louis, Mo.). Non-essential amino acids, penicillin and streptomycin, and L-glutamine were purchased from Life Technologies Inc. (Carlsbad, Calif.). The membrane permeable PKA inhibitor, myristoylated PKI was purchased from Calbiochem (San Diego, Calif.). The calpain inhibitor E64 was purchased from Roche Molecular Biochemicals (Indianapolis, Ind.). Goat anti-mouse immunoglobulin (IgG) conjugated with horseradish peroxidase (HRP), FITC-conjugated goat anti-mouse IgG were purchased from Biosource (Camarillo, Calif.).

Recombinant GPIb-IX and mutants-CHO cells expressing recombinant wild type GPIb-IX, a GPIb-IX mutant with a serine to alanine point mutation at Ser166 in GPIbβ (S¹⁶⁶A), and GPIb-IX mutants with truncations at 591 and 605 in the cytoplasmic domain of GPIbα were described previously (Bodnar et al., J. Biol. Chem., 277(49):47080-47087 (2002); Du et al., J. Biol. Chem., 271:7362-7367 (1996)). Site directed mutagenesis that replaces Ser 609 of GPIbα to an alanine (S609A) was performed using PCR method with the forward primer as AGAAGAATTCGCTGCTCTGACCACA (SEQ ID NO:5) and the reverse primer as TAAGTCTAGATCAGAGGGCGTGGCCAGAGT (SEQ ID NO:6). The PCR product was cloned into TA vector (Invitrogen), and inserted into wild type GPIbαin pGEM3Z(+) vector after digestion with restriction enzymes Sma I and BamH I. The resulting S609A mutant were then subcloned into pcDNA3.1(−) vector after digestion with EcoR I. Correct mutation was verified by DNA sequencing. Transfection of cDNA into CHO cells was performed using LipofectAMINE Plus (Invitrogen) as recommended by the manufacturer. Stable cell lines were selected using selection media containing 0.5 mg/ml G418 and further selected by cell sorting using the anti-GPIbα monoclonal antibody, SZ2. Expression of GPIb-IX in different cell lines was adjusted by cell sorting to comparable levels before experiments.

Flow Cytometric Analysis of VWF Binding to GPIb-IX-expressing Cells—CHO cells expressing wild type (1b9) and mutant GPIb-IX were grown to confluence to synchronize cell growth. The cells were detached using 0.5 mM EDTA in PBS, pH 7.4. Seventy-five percent of the original volume was then replated in Dulbecco's modified Eagle's medium (DMEM) growth media and cultured for 18 h. For VWF binding assays, cells were detached using 0.5 mM EDTA-PBS, pH 7.4, resuspended to a concentration of 2.25×10⁶ cells/ml and incubated in modified Tyrode's buffer (2.5 mM Hepes, 12.1 mM NaHCO₃, 2.36 mM KCl, 0.136 M NaCl, 1 mM CaCl₂, 1 mM MgCl₂, 0.1% D-glucose, 1% BSA, pH 7.4) for 30 min at 4° C. Ristocetin (1.25 mg/ml) and purified vWF (35 μg/ml) were added to cell suspension and incubated at 22° C. for 30 min. After washing once with Tyrode's buffer, cells were further incubated with 10 μg/ml FITC-labeled SZ-29 (a monoclonal antibody against vWF) in the dark at 22° C. for 30 min and then analyzed by flow cytometry. In some experiments, VWF were preincubated with FITC-labelled SZ29 and then allowed to incubate with cells for 30 min.

The cells were then analyzed by flow cytometry without washing. Results obtained with these two different VWF binding methods were very similar. As negative controls, cells were incubated with ristocetin in the absence of VWF. To examine the effects of myristoylated PKI, cells were preincubated with or without 200 μM PKI for 15 min at 22° C., prior to analysis for VWF binding.

Cell Adhesion under Flow—Purified human vWF was diluted to a final concentration of 30 μg/ml with 0.1 mM NaHCO3, pH 8.3, and coated onto glass capillary tubes (inner diameter 0.58 mm, Harvard Apparatus Inc.) overnight in a humid environment at 4° C. The cover capillaries were washed with PBS to remove unbound vWF, blocked with 5% BSA in PBS at room temperature for 2 hrs, and then installed on the stage of an inverted microscope. The cells were suspended in modified Tyrode's buffer containing 0.5% BSA (5×10⁶ cells/mL), and then perfused by a syringe pump (PhD, Harvard Apparatus Inc.) into the glass capillaries at various shear rates for 2.5 min. Shear rate was calculated as described by Slack and Turitto (Slack et al., Thromb. Haemost., 72(5):777-781 (1994)). Transient adhesion (rolling) of cells on the vWF-coated surface was recorded on video cassette recorder, and the rolling cells were counted in 10 randomly selected fields and at randomly selected time points.

Communoprecipitation of GPIb-IX with 14-3-3-CHO cells expressing wild type and mutant GPIb-IX were resuspended in Hepes buffer (137 mM NaCl, 2.7 mM KCl, 1 mM MgCl₂, 5.6 mM D-glucose, 3.3 mM Na₂HPO₄, 3.8 mM Hepes, pH 7.35) to a concentration of 1.0×10⁸ cells/ml then solubilized by adding an equal volume of solubilization buffer (0.1 M Tris, 0.01 M EGTA, 0.15 M NaCl, and 2% Triton X-100, pH 7.4) containing 0.2 mM E64 and 1 mM phenylmethylsulfonyl fluoride (PMSF), 1 mM DTT, and 0.08 U/mL aprotinin. The samples were centrifuged at 100,000×g at 4° C. for 30 min to remove the Triton X-100 insoluble material. Cell lysate (150 μl) was incubated with anti-GPIbα monoclonal antibody P3 for 1 hour, and then with protein G-conjugated Sepharose 4Bbeads for 1 hour at 4° C. The beads were then washed three times with 1:1 mix of Hepes buffer and lysis buffer. Bound proteins were extracted by the addition of SDS-sample buffer and immunoblotted with an anti-14-3-34 antibody (Gu et al., J. Biol. Chem., 273(50):33465-33471 (1998)).

Association of GPIb-IX with Triton X-100-insoluble membrane skeleton—CHO cells expressing wild type or mutant GPIb-IX were resuspended in Tyrode's buffer with 0.1% BSA to a concentration of 1.0×10⁷ cells/ml. The cells were incubated at 4° C. for 30 min, then solubilized in an equal volume of lysis buffer containing 0.2 mM E64, 2 mM PMSF, and 0.08 U/ml aprotinin for 30 min at 4° C. The lysate was centrifuged at 2000×g for 5 min to remove the nuclei. The cell lysates were then centrifuged at 100,000×g for 3 hours at 4° C. The insoluble pellet and supernatant were dissolved in identical final volumes of SDS-sample buffer containing 5% β-mercaptoethanol, and then Western blotted for GPIbα using the monoclonal antibody, MW23.

### Results

There are two known phosphorylation sites in the cytoplasmic domain of GPIb-IX. Ser609 in the vast majority of GPIbα molecules is constitutively phosphorylated in platelets (Bodnar et al., J. Biol. Chem., 274(47):33474-33479 (1999)). Phosphorylation at this site is required for high affinity binding of 14-3-3 to the C-terminal domain of GPIbα. Ser166 of GPIbβ is phosphorylated by cAMP-dependent protein kinase (PKA), an event dynamically regulated by intracellular cAMP level (Fox et al., J. Biol. Chem., 262(26):12627-12631 (1987); Fox et al., J. Biol. Chem., 264(16):9520-9526 (1989)). Phosphorylation at Ser166 is important in 14-3-3 interaction with the cytoplasmic domain of GPIbβ (Andrews et al., Biochemistry, 37(2):638-647 (1998); Calverley et al., Blood, 91(4):1295-1303 (1998); Feng et al., Blood, 95(2):551-557 (2000)). PKA-dependent phosphorylation at Ser166 of GPIbβ negatively regulates vWF binding function of GPIb-IX (Bodnar et al., J. Biol. Chem., 277(49):47080-47087 (2002)). Thus, dephosphorylation of Ser166 enhances vWF binding function of GPIb-IX. To further investigate the roles of serine phopshorylation in regulating GPIb-IX function, cell lines were established that express following mutants of GPIb-IX: (1) GPIb-IX with wild type GPIbβ and GPIX complexed with a GPIbα-mutant bearing a conserved mutation of Ser609 to an alanine residue (S609A), (2) GPIb-IX with wild type GPIbα and GPIX complexed with a mutant of GPIbβ in which Ser166 is mutated to an alanine (S166A), and (3) GPIb-IX bearing both GPIbα S609A and GPIbβ S¹⁶⁶A mutations (S166A/S609A). By repeated cell sorting using anti-GPIb monoclonal antibodies, cells lines with expression levels of these different mutants comparable to a wild type GPIb-IX cell line (1b9) were obtained.

To determine the roles of the above described two phosphoserine residues in regulating ligand binding function of GPIb-IX, CHO cell lines bearing wild type and the mutants of GPIb-IX were incubated with purified VWF in the presence of ristocetin (a VWF modulator that induces VWF binding to GPIb-IX). Binding of VWF was determined by flow cytometry using an anti-VWF monoclonal antibody. As reported previously (Englund et al., J. Biol. Chem., 276(20):16952-16959 (2001); Bodnar et al., J. Biol. Chem., 277(49):47080-47087 (2002)), only a low level of VWF binding to wild type GPIb-IX expressed in CHO cells (1b9) was detected. This is because GPIb-IX molecules expressed in CHO cells are phosphorylated at Ser166 and thus in a “resting” form (Bodnar et al., J. Biol. Chem., 277(49):47080-47087 (2002)). In contrast, vWF binding to cells expressing S¹⁶⁶A mutant was significantly higher, indicating that this mutant has an enhanced vWF binding function. GPIbα S609A mutation by itself has no significant effect on vWF binding function as indicated by similar levels of VWF binding compared to wild type GPIb-IX expressing cells. However, when S609A mutant of GPIbα were complexed with S166A mutant of GPIbβ, the enhancing effect of S166A on VWF binding was diminished. Thus, the phosphoserine 609 is required for the enhancing effects of S166A mutation on vWF binding function.

GPIb-IX binding to vWF mediates transient adhesion (or rolling) of platelets on the subendothelial-bound vWF under flow conditions. Therefore, studies were performed to determine whether Ser609 mutation in the GPIbα C-terminal 14-3-3 binding also affect GPIb-IX-dependent cell adhesion to vWF under flow conditions. It was seen that a very low number of CHO cells expressing wild type GPIb-IX (1b9) were able to adhere and roll on vWF-coated surface at a shear rate of 250 s−1. In contrast, the S166A mutant cells showed a significantly enhanced adhesion. This enhancing effect of S166A mutation was diminished in S166A/S609A cells, indicating that the phosphoserine 609 is required in S166A mutation-induced activation of cell adhesion mediated by GPIb-IX.

The S¹⁶⁶A mutation of GPIbβ is highly conserved because the only difference between serine and alanine is the presence of a hydroxyl group, which serves as the phosphorylation site. Since Ser166 of wild type GPIbβ expressed in CHO cells is known to be phosphorylated, it is likely that the effects of S¹⁶⁶A mutant on enhancing vWF binding function of GPIb-IX result from abrogation of Ser166 phosphorylation. Therefore, the data that GPIbα S609A mutation abolishes the enhancement of vWF binding function by S166A mutation suggest the possibility that GPIbα phosphoserine 609 is required for Ser166-dephosphorylation induced upregulation of vWF binding function of GPIb-IX. A specific membrane permeable PKA inhibitor, the myristoylated PKA inhibitor peptide (PKI), has been shown to cause dephosphorylation of GPIbβ Ser166 and thus enhances VWF binding function of GPIb-IX (Bodnar et al., J. Biol. Chem., 277(49):47080-47087 (2002)). Thus, the inventors further examined whether the S609A mutation of GPIbα could also reverse the enhancing effect of PKI on vWF binding to GPIb-IX. To do this, cells expressing wild type or S609A mutant of GPIb-IX were preincubated with PKI and then examined for ristocetin-induced vWF binding. Previously, it has been shown that PKI treatment significantly reduced phosphorylation at Ser166 of GPIbβ, but has no significant effect on GPIbα Ser609 phosphorylation (Bodnar et al., J. Biol. Chem., 277(49):47080-47087 (2002); Bodnar et al., J. Biol. Chem., 274(47):33474-33479 (1999)). PKI treatment significantly enhanced vWF binding to wild type GPIb-IX (1b9 cells), but this effect was dramatically reduced in the S609A mutant. This result indicates that phosphoserine 609 is important in Ser166-dephosphorylation-induced enhancement of vWF binding to GPIb-IX.

Phosphoserine 609 is a key residue in the high affinity binding of 14-3-3 to GPIbα. Thus, the effect of S609A mutation is likely to result from a loss of 14-3-3 binding. To determine if deletion of entire 14-3-3 binding site in the C-terminal domain of GPIbα would also have effects similar to S609A, the effects of PKI on vWF binding function of two different mutants of GPIb-IX, A591 and A605, lacking the GPIbαC-terminal 20 and 5 residues respectively was examined. PKI-induced upregulation of vWF binding was inhibited in Δ591 and Δ605 mutants, suggesting that the 14-3-3 binding region in the C-terminus of GPIbα is important in the GPIbβ phosphorylation (dephosphorylation)-induced dynamic regulation of VWF binding function of GPIb-IX.

Both Phosphoserine 609 of GPIbα and phosphoserine 166 of GPIbβ have been shown to be the key residues in 14-3-3 binding sites. Thus, it is likely that the effects of S166A and S609A mutants on VWF binding function of GPIb-IX result from loss of 14-3-3 binding to GPIbα and GPIbβ respectively. To determine whether these mutants affect endogenous 14-3-3 binding to GPIb-IX, 1b9, S609A, S166A and S609A/S609A cells were solubilized and immunoprecipitated with an anti-GPIbα antibody. The immunoprecipitates were then immunoblotted with an anti-14-3-3 antibody.

The results showed that 14-3-3 co-immunoprecipiated with wild type GPIb-IX and also with S166A mutant, suggesting that S166A mutation is not sufficient to reduce 14-3-3 binding. In contrast, 14-3-3 failed to co-immunoprecipitate with S609A mutant or S609A/S166A mutants. Thus, although there are 3 different 14-3-3 binding sites in the cytoplasmic domain of GPIb-IX complex, the site containing the phosphoserine 609 is required for high affinity interaction between GPIb-IX and endogenous 14-3-3 under these conditions. These data also suggest that the 14-3-3 binding site in the cytoplasmic domain of GPIbβ, by itself, is not sufficient to support the high affinity interaction between GPIb-IX and 14-3-3. Hence, anchoring of 14-3-3 to the C-terminal domain of GPIbα may facilitate the potential interaction between the dimeric 14-3-3 with GPIbβ or other sites in the cytoplasmic domains of GPIb-IX.

It is known that the cytoplasmic domain of GPIbα interacts with filamin and thus links GPIb-IX to short actin filamental structure underlying the membrane, which is referred to as “the membrane skeleton”. The association of GPIb-IX with the membrane skeleton negatively regulates VWF binding function of GPIb-IX. The present example demonstrates that disruption of Ser166 phosphorylation enhances VWF binding to GPIb-IX by a mechanism that requires 14-3-3 binding site in the C-terminal domain of GPIbα. To determine the relationship between these two seemingly different regulatory mechanisms of VWF-GPIb-IX interaction, the association of wild type and different mutants of GPIb-IX with the membrane skeleton were examined. These studies showed that the majority of wild type GPIb-IX molecules is associated with Triton X-100 insoluble membrane skeleton fraction that can be sedimented at 100,000 g. In contrast, there is a significant fraction of S¹⁶⁶A mutant present in the soluble fraction. When GPIbβ S166A mutant is complexed with GPIbα S609A mutant (S166A/S609A), soluble fraction was reduced compared with S166A mutant. These data suggest that dephosphorylation of GPIbβ at Ser166 made GPIb-IX more likely to dissociate from the membrane skeleton, which was shown to enhance the vWF binding function of GPIb-IX. Taken together, these data suggest that phosphorylation of Ser609 of GPIbα and phosphorylation-dependent binding of 14-3-3 to the C-terminal site of GPIbα is required for Ser166-dephosphorylation-induced dissociation of GPIb-IX from the membrane skeleton and subsequent upregulation of VWF binding function of GPIb-IX.

### Discussion

In the present example, it is shown that the key residue in GPIbα C-terminal 14-3-3 binding site, phosphoserine-609, is required in upregulating vWF binding function of GPIb-IX induced by dephosphorylation of Ser166, and is also required for GPIbβ dephosphorylation-induced regulation of GPIb-IX association with the membrane skeleton.

The results that phosphorylation of GPIbβ Ser166 and GPIbα Ser609 regulates ligand binding function of GPIb-IX suggest a role for the phosphoserine-dependent signaling molecule, 14-3-3, in regulating ligand binding function of GPIb-IX. Among the identified 14-3-3 binding sites, only the GPIbβ interaction with 14-3-3 is known to be dynamically regulated by PKA-dependent phosphorylation at Ser166 (Wardell et al., J. Biol. Chem., 264(26):15656-15661 (1989)). Thus, the data that phosphorylation at Ser166 down regulates vWF binding to GPIb-IX suggests that interaction between 14-3-3 and GPIbβ, which is enhanced by Ser166 phosphorylation, negatively regulates ligand binding function of GPIb-IX. Conversely, dephosphorylation of Ser166 appears to upregulate VWF binding by disruption of 14-3-3 binding to GPIbβ. Furthermore, these data suggest that upregulation of vWF binding function induced by Ser166 dephosphorylation requires phosphorylation-dependent binding of 14-3-3 to GPIbα C-terminal binding site, because disruption of the C-terminal 14-3-3 binding site in GPIbα reversed the Ser166 dephosphorylation-induced upregulation of VWF binding. Thus these results suggest that intracellular signals, by controlling PKA activity, regulate ligand binding function of GPIb-IX by modulating 14-3-3 binding states of GPIb-IX.

The 14-3-3 protein is dimeric. Each monomer of the 14-3-3 dimer has a binding pocket, and thus the dimeric 14-3-3 is able to simultaneously interact with two different sites of a protein or two different ligands (Liu et al., Nature, 376(6536):191-194 (1995); Xiao et al., Nature, 376(6536):188-191 (1995); Braselmann et al., EMBO J. 14(19):4839-4848 (1995)). Therefore, it is possible that different 14-3-3 binding sites in GPIb-IX may interact with the single 14-3-3 dimer. It has previously shown that high affinity binding of 14-3-3 to GPIb-IX requires GPIbα C-terminal 14-3-3 binding site (Bodnar et al., J. Biol. Chem., 274(47):33474-33479 (1999)), in which the constitutively phosphorylated Ser609 plays a key role. It has also been shown that binding of monomeric 14-3-3 to GPIb-IX is significantly lower than dimeric 14-3-3, suggesting that high affinity binding of dimeric 14-3-3 to GPIb-IX involves simultaneous interaction with two 14-3-3 binding sites (Gu et al., J. Biol. Chem., 273(50):33465-33471 (1998)). PKA-dependent phosphorylation of Ser166 forms a high affinity 14-3-3 binding site in GPIbβ. Thus it is likely that a 14-3-3 dimer can interact with both GPIbα C-terminal and GPIbβ 14-3-3 binding sites when GPIbβ is phosphorylated at Ser166. Conversely, dephosphorylation of GPIbβ Ser166 reduces 14-3-3 affinity for GPIbβ under which conditions 14-3-3 only interact with the GPIbα cytoplasmic domain. This interaction is required for the upregulation of vWF binding function induced by Ser166 dephosphorylation. Thus, while there are alternative possibilities, current data suggest that PKA-mediated switch between a 14-3-3 dimer binding to both GPIbα and GPIbβ and 14-3-3 binding to GPIbα alone determines whether GPIb-IX is in a “resting” state, or an “active” state. Therefore, the inventors propose a “toggle switch” model as a possible regulatory mechanism of GPIb-IX function. As discussed above, simultaneous occupation of two binding sites in GPIb-IX cytoplasmic domain are important in high affinity binding of 14-3-3, and one of the binding site must be the GPIbα C-terminal RYSGHpS609L sequence. While PKA-phosphorylated GPIbβ can serve as the other binding site, deletion of GPIbβ 14-3-3 binding site or S166A mutation do not reduce 14-3-3 binding to GPIb-IX, suggesting that, when 14-3-3 binding site in GPIbβ is disrupted, 14-3-3 may still interact with two 14-3-3 binding sites. Since there is evidence indicating the presence of a second 14-3-3 binding site in the central region of GPIbα cytoplasmic domain (Andrews et al., Biochemistry, 37(2):638-647 (1998); Feng et al., Blood, 95(2):551-557 (2000)), it is possible that a 14-3-3 dimer simultaneously interact with two binding sites in GPIbα when GPIbβ is dephosphorylated, which switches on the ligand binding function of GPIb-IX. PKA-mediated phosphorylation of GPIbβ allows high affinity interaction of 14-3-3 with both GPIbαand GPIbβ, switching off ligand binding function.

Interestingly, the second binding site for 14-3-3 in the central region of GPIbα overlaps with filamin binding site (Andrews et al., J. Biol. Chem., 267(26):18605-18611 (1992); Andrews et al., Biochemistry, 37(2):638-647 (1998); Feng et al., Blood, 102(6):2122-2129 (2003)), suggesting a possibility that 14-3-3 interaction with this region of GPIbα may interfere with GPIb-IX association with the membrane skeleton. This possibility is supported by data that disruption of Ser166 phosphorylation site by S166A mutation decreases percentage of GPIb-IX molecules that are associated with the Triton X-100-insoluble membrane skeleton, and that the effect of S166A mutation on GPIb-IX association with the membrane skeleton is significantly attenuated by disruption of the C-terminal 14-3-3 binding site in GPIbα. Thus, it is possible that the 14-3-3 “toggle switch” regulates GPIb-IX association with the membrane skeleton actin filaments. Since previous studies show that dissociation of GPIb-IX with the membrane skeleton enhances VWF binding function (Englund et al., J. Biol. Chem., 276(20):16952-16959 (2001)), it is possible that the 14-3-3 “toggle switch”, by controlling the association of GPIb-IX with the membrane skeleton, regulates vWF binding function of GPIb-IX.

### Example 4

In order to evaluate the in vivo anti-thrombotic effect of MPUC, a micelle formulation of MPαC was developed, which can be safely used in in vivo treatment and experiments. In the following experiments, myristoylated peptides, MPαC(C₁₃H₂₇CONH-SIRYSGHpSL), were mixed with PEG2000-DSPE (Northern Lipids Inc. Vancouver, BC), L-α-phosphatidylcholine (egg PC, Type XI-E) (Sigma-Aldrich, St. Louis, Mo.) and at the molar ratio of 1:45:5 under conditions giving rise to the desired micellar composition.

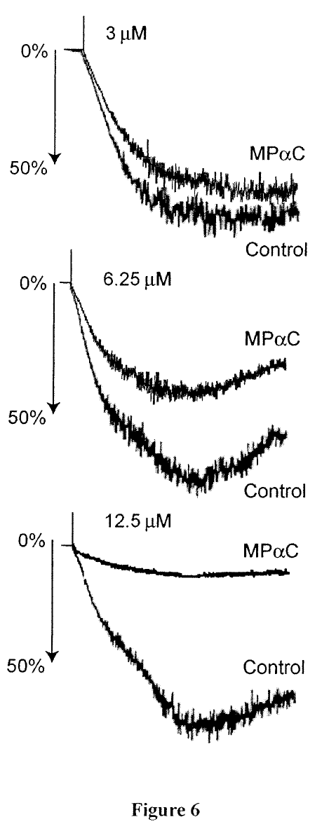

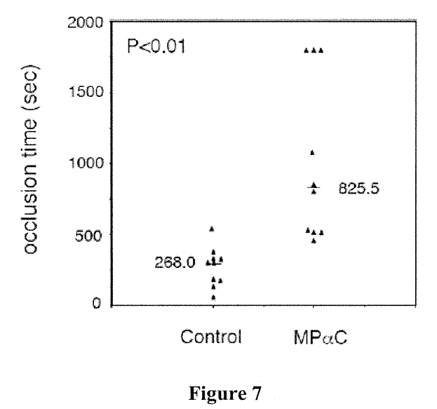

The micellar MPαC inhibited ristocetin induced platelet agglutination (FIG. 6) in a dose dependent manner, indicating that it is an effective inhibitor of the VWF/GPIb-IX interaction. Pretreatment of mice with micellar MPαC caused significant (P<0.01) delayed occlusive thrombus formation in the FeCl₃-injured carotid artery thrombosis model, in comparison with the scrambled peptide control, indicating that micellar MPαC is an effective anti-thrombotic agent (FIG. 7).

### Example 5

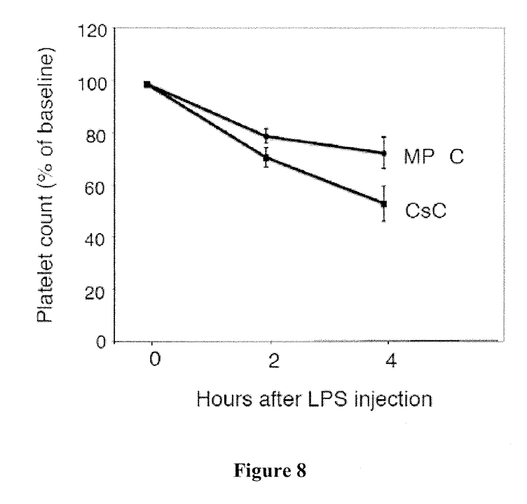

To investigate the role of GPIb-IX in bacteria endotoxin lipopolysaccharide (LPS)-induced microvascular thrombosis, thrombocytopenia and mortality, the therapeutic effect of the micellar MPαC in a LPS-induced mouse sepsis model was examined. Results showed that mice developed thrombocytopenia following LPS challenge (12 mg/kg body weight) in a time-dependent manner (FIG. 8), and LPS-induced thrombocytopenia was significantly (P<0.01) mitigated by MPαC pretreatment (FIG. 8).

### Example 6

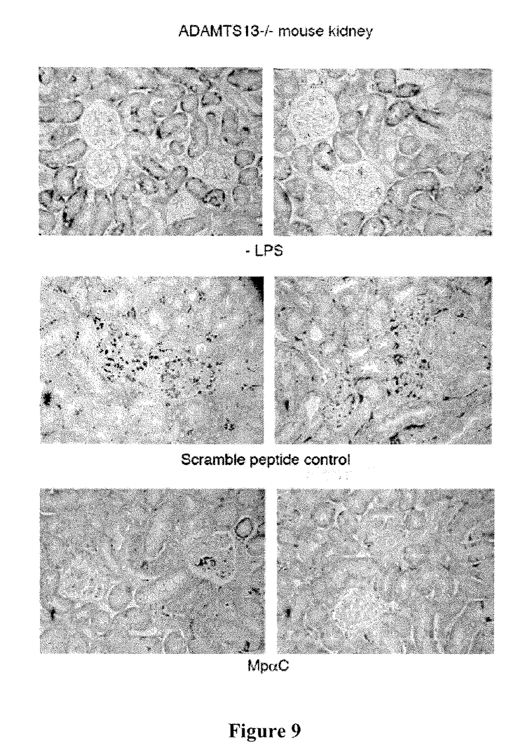

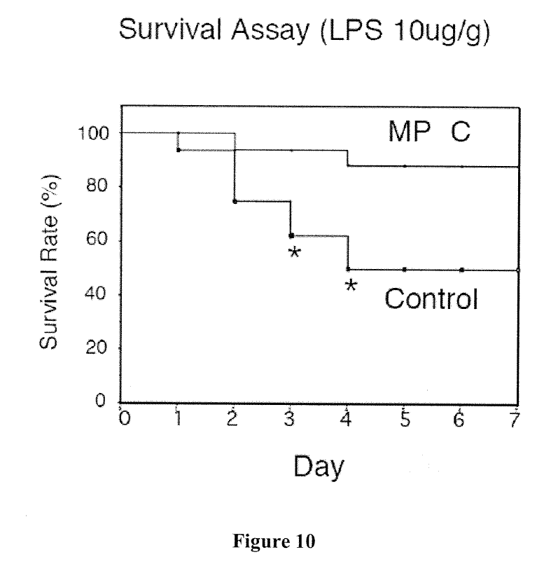

Pathohistology studies revealed that LPS (30 mg/kg body weight) treatment induced microvascular thrombus formation in the kidney of ADAMTS13 knockout mice (FIG. 9). Micellar MPαC pretreatment partially but significantly inhibited LPS-induced microvascular thrombosis (FIG. 9). Importantly, a single dose of micellar MPαC injection significantly improved survival rate (P<0.05) of LPS-treated wild type mice after LPS challenge compared to scrambled control peptide (FIG. 10).

These data show that the platelet VWF receptor GPIb-IX plays an important role in LPS-induced microvascular thrombosis, thrombocytopenia, and mortality. The results also demonstrate that micellar MPαC is a new agent that is effective in treating LPS-induced sepsis by mitigating microvascular thrombosis and thrombocytopenia.

### Example 7

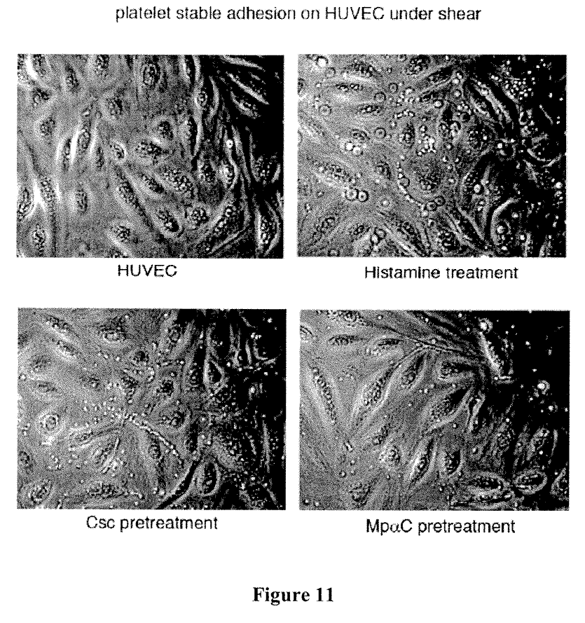

To investigate whether the therapeutic effect of MPαC in treating microvascular thrombosis results from its effect on preventing platelet adhesion to vascular endothelial cells, human umbilical vascular endothelial cells (HUVEC) were cultured to full confluency and stimulated with inflammatory agonist histamine. Washed platelets were then exposed to endothelial cells under the shear rate of 800s⁻¹. Results showed than platelets adhered to histamine-treated endothelial cells and this adhesion was inhibited by MPαC (FIG. 11). Thus, the effect of MPαC in mitigating platelet adhesion to vascular endothelial cells is a mechanism for the therapeutic effect of MPαC in endotoxemia.

Combined, these data show that micellar MPαC is effective in (a) inhibiting arterial thrombosis in vivo; (b) inhibiting microvascular thrombosis induced by LPS in endotoxemia; (c) relieving thrombocytopenia in endotoxemia; (d) improving survival rates in endotoxemia in a mammal.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

What is claimed is:

1. A method of treating or preventing thrombosis in a mammal comprising the step of administering an inhibitor of GPIb-IX binding to von Willebrand factor selected from the group consisting of a blocking antibody, soluble GPIb-IX fragments, DNA aptamers, and peptides or small molecule compounds that block VWF-GPIb-IX interaction in an amount effective for treating thrombosis, said inhibitor comprising a coating.

2. The method of claim 1 wherein said thrombosis is arterial thrombosis.

3. The method of claim 1 wherein said thrombosis is microvascular thrombosis.

4. The method of claim 1 wherein said thrombosis is associated with sepsis.

5. The method of claim 1 wherein said thrombosis is associated with endotoxemia.

6. The method of claim 1 wherein the coating is a surfactant.

7. The method of claim 6 wherein the surfactant is a lipid.

8. The method of claim 7 wherein the lipid is lecithin.

9. The method of claim 1 wherein the coating is a micelle.

10. The method of claim 1 wherein the mammal suffers from thrombotic thrombocytopenia or acquired microangiopathy.

11. The method of claim 1 wherein the survival rate of the mammal is increased compared to a mammal suffering from thrombosis and not administered said inhibitor.

12. The method of claim 1 wherein the mammal suffers from hemolytic uremic syndrome.

13. The method of claim 1 wherein the mammal is a human.

14. A composition comprising a peptide having an amino acid sequence of:
a. SIRYSGHpSL (SEQ ID NO: 1)
b. a peptide fragment of SEQ ID NO: 1 that retains a 14-3-3 binding activity,
c. or conservative peptide variant of SEQ ID NO: 1 that retains a 14-3-3 binding activity,
wherein said peptide of a, b or c has a myristoyl group C₁₃H₂₇CONH— at one or both of C-terminus or N-terminus of the peptide, said composition further comprising a coating.

15. The composition of claim 14 wherein the coating is a surfactant.

16. The composition of claim 15 wherein the surfactant is a lipid.

17. The composition of claim 16 wherein the lipid is lecithin.

18. The composition of claim 14 wherein the coating is a micelle.
